(19) United States
(12) Patent Application Publication
(10) Pub. No.: US 2024/0029878 A1
(43) Pub. Date: Jan. 25, 2024

(54) COMPREHENSIVE PATIENT BIOLOGICAL INFORMATION COLLECTION DEVICES AND EMERGENCY RESPONSE SYSTEMS USING THE SAME

(71) Applicant: XIAO JUN WEN, NORCROSS, GA (US)

(72) Inventor: XIAO JUN WEN, NORCROSS, GA (US)

(21) Appl. No.: 17/869,599

(22) Filed: Jul. 20, 2022

(51) Int. Cl. G16H 40/67; G16H 80/00; H04L 67/12; H04W 4/029; H04W 4/90

(52) U.S. Cl. CPC G16H 40/67; G16H 80/00; H04L 67/12; H04W 4/029; H04W 4/90

(57) ABSTRACT

The present disclosure relates to a comprehensive patient biological information collection device (CPBICD). Each patient carries one CPBICD. The CPBICD uses a set of patient biological information collection sensors to collect and monitor patient's biological information constantly. In certain embodiments, CPBICD is used to monitor patients' wellbeing in an emergency response system. Biological information of each patient is collected and transmitted to CPBICD server for processing using artificial intelligence algorithms to detect early emergency signs before emergency occurs. When at least one of the patient biological information collection sensors detects certain type of biological information that exceeds a normal range of baseline biological information stored in the patient database, emergency response system determines the patient is near or in an emergency, and coordinates immediate medical assistance to the patient based on the patient information received and retrieved from the patient database.

### FIELD

The present disclosure generally relates to medical devices, and more particularly to comprehensive patient biological information collection devices, emergency response systems using the comprehensive patient biological information collection devices, and the methods of using the emergency response systems.

### BACKGROUND

According to World Health Organization (WHO), of the 56.9 million deaths worldwide in 2016, more than half (30.7 million or 54%) were due to these top three causes related to heart conditions, including ischemic heart diseases, stroke, and chronic obstructive pulmonary. Ischemic heart disease, stroke and chronic obstructive pulmonary are the world's biggest killers, accounting for a combined 18.2 million deaths in 2016. These diseases have remained the leading causes of death globally in the last 15 years.

Majority of deaths from ischemic heart disease and stroke are preventable if the patients receive care and take the appropriate medications immediately; or if the early symptoms of heart attacks and/or stroke are detected and recognized, and preventive treatments are given before stroke or heart attack. The most important things for a patient to survive from medical emergencies include three key components, early signs recognition or detection to prevent emergency from happening; patients, medical professionals, emergency responders, and care takers are immediately alerted or noticed; and patients, medical professionals, emergency responders, and care takers work together to respond promptly to provide the appropriate care needed to survive or avoid serious complications.

Thus, it is desirable to have an Emergency Signs Early Detection, Alert and Response System to ensure: the early signs or precursors of a medical emergency can be detected as early as possible to prevent it from happening, the patient receives immediate medical care by trained professionals, the patient receives and takes appropriate medications, and nearby emergency dispatch centers are notified and ambulance and medical emergency staff arrive at the scene in shortest possible time.

Currently, there is no ESEDARS in present market. To detect the early warning signs of emergency conditions, such as strokes and heart attacks, several vital signs must be measured or monitored, including, blood pressure, heart beat rate, pulse, body temperature, respiratory rate, blood sugar, and EKG etc. Medical devices are widely available for home use and affordable for ordinary families to collect the key biological or vital information as described above. However, there are still significant challenges or deficiencies in using those standalone medical devices needed to detect early emergency signs: 1) lack of a comprehensive software and hardware system that makes the collections, storage, and analysis of the data collected by standalone devices reliable and useful during the emergency situations; 2) it is not user-friendly to use multiple standalone medical devices to collect, transmit, store and use the data; 3) costs are much higher to manufacture standalone medical devices with their own hardware and software for the data collection, procession and analysis than a comprehensive one; and 4) waste of resources: while a comprehensive patient biological informational collection device (CPBICD) can share the same hardware and software for the data processions, transmission, storage and analysis to significantly reduce the waste of resources and costs of manufacture. It is desirable to have a user-friendly, low-cost, and resource-saving alternative to monitor patients' biological information from multiple sensors in one comprehensive system to make the data collections, transmissions, storage, and analysis much easier and the information collected more reliable and useful.

Therefore, a heretofore unaddressed needs still exist in the art to address the aforementioned deficiencies and inadequacies.

### SUMMARY

In one aspect, the present disclosure relates to a comprehensive patient biological information collection device. In certain embodiments, the comprehensive patient biological information collection device collects a patient's biological information through a set of patient biological information collection sensors and monitors the wellbeing of the patient. The comprehensive patient biological information collection device includes: a processor, a network interface controller, and a non-volatile memory. The processor controls operations of the comprehensive patient biological information collection device. The network interface controller facilitates the communication among the comprehensive patient biological information collection device, and the set of patient biological information collection sensors over a communication network. The non-volatile memory stores an operating system, a GPS module for detecting the GPS location of the patient carrying the comprehensive patient biological information collection device, and a comprehensive patient biological information collection device controller having a patient communication module, a comprehensive patient biological information collection module, a comprehensive patient biological information processing module, a comprehensive patient biological information storage module; and computer executable instructions. When executed by the processor, the computer executable instructions perform one or more of following operations:
receiving, through the comprehensive patient biological information collection module over the patient communication module, patient biological information constantly monitored and collected by a set of patient biological information collection sensors;
processing, by the comprehensive patient biological information processing module, the patient biological information received from the set of patient biological information collection sensors;
storing, by the comprehensive patient biological information storage module, the processed patient biological information; and
updating, by the patient communication module, a patient database.

In certain embodiments, the patient communication module facilitates communication among the patient database, the comprehensive patient biological information collection device controller, and the set of patient biological information collection sensors through the network interface controller over the communication network. The comprehensive patient biological information collection module collects patient biological information through a wireless data receiver over the communication network from the set of patient biological information collection sensors from the patient. The comprehensive patient biological information processing module processes the patient biological information collected through the comprehensive patient biological information collection module. The comprehensive patient biological information storage module stores the patient biological information processed through the comprehensive patient biological information processing module and synchronizes the patient biological information stored with the patient database.

In certain embodiments, the communication network includes a wireless personal area network (WPAN) having a Wi-Fi network, a Bluetooth network, an infrared network, and a Zigbee network, a wireless local area network (WLAN), a wireless metropolitan area network (WMAN), a wireless wide area network (WWAN), a cellular network, and a mobile communication network.

In certain embodiments, the set of patient biological information collection sensors includes one or more of:
a chip reader for reading a patient information card where the patient information is stored;
a geolocation sensor for monitoring the geolocation of the patient;
a speaker for the patient to make and receive voice calls, and to provide voice instructions to the patient;
a stethoscope sensor for monitoring patient's heart, lung and breathing conditions;
a blood oxygen sensor for monitoring patient's blood oxygen level;
a motion sensor for monitoring patient's movement;
an acoustic sensor for making and receiving voice calls by the patient, generating voice samples of the patient, and detecting background sounds surrounding the patient;
a temperature sensor for monitoring patient's body temperature;
a blood pressure sensor for monitoring patient's blood pressure;
a blood sugar sensor for monitoring patient's blood sugar level;
an EKG/ECG sensor for monitoring patient's electrocardiograms;
an EEG sensor for monitoring patient's electroencephalograms;
a video camera for monitoring patient's posture, movement, facial and emotional expressions;
an accelerometer for detecting impact to the patient and sudden fall of the patient;
an abnormal gas sensor for detecting harmful gas around the patient; and
a breathalyzer sensor for detecting blood alcohol level of the patient.

In another aspect, the present disclosure relates to an emergency response system. The emergency response system can be triggered either by a patient pressing an emergency button when a patient realizes that he/she may be in an emergency situation, or by a comprehensive patient biological information collection device where the patient carries comprehensive patient biological information collection device, and the comprehensive patient biological information collection device constantly monitors one or more patient biological information, and triggers emergency response system when the comprehensive patient biological information collection device detects certain type of biological information that exceeds a normal range of the set of baseline biological information stored in the patient database.

The emergency response system can also be triggered remotely either by an emergency responder or a healthcare provider who is registered in the system and with a specialized App of this system installed in any of his/her smart devices, such as a smart phone, to collect the biological or medical information from, to give medical advices given to, and establish communication channels with the patient in need for help who carries or has access to the comprehensive patient biological information collection device.

In certain embodiments, the emergency response system includes: an emergency response system server, a communication network, a communication system, a patient database, and a set of comprehensive patient biological information collection devices. Each patient carries one comprehensive patient biological information collection device. Each patient and a corresponding comprehensive patient biological information collection device are registered at the emergency response system server. The emergency response system provides immediate emergency assistance to the patient when the corresponding comprehensive patient biological information collection device of the patient detects one or more emergency signs from the patient.

In certain embodiments, the patient database is connected to and accessible by the emergency response system server. The patient database stores patient information of all patients. The patient information includes personal information, medical history, a set of baseline biological information, patient contact information, and contact information of relatives and friends and local medical facilities to be notified. The communication system is also connected to the emergency response system server. The communication system provides voice, text, and video communication over the communication network among the patients, one or more live emergency responders from a nearby emergency dispatch center, one or more patient's relatives and friends on record, and one or more nearby medical facilities to provide immediate emergency assistance to the patient when emergency occurs.

In certain embodiments, each comprehensive patient biological information collection device collects patients' biological information through a set of patient biological information collection sensors and the set of patient biological information collection sensors constantly monitors the biological information of the patient and transmits the patient biological information collected to the emergency response system server through the comprehensive patient biological information collection device. When at least one of the patient biological information collection sensors detects certain type of biological information that exceeds a normal range of the set of baseline biological information stored in the patient database, the emergency response system server initiates at least voice communication between the emergency response system server and the comprehensive patient biological information collection device of the patient through the communication system directly. Each comprehensive patient biological information collection device also includes an emergency medicine storage for storing one or more patient specific emergency medicines to be used when emergency occurs.

In certain embodiments, a patient emergency indicator of the comprehensive patient biological information collection device is lit in green light indicating the comprehensive patient biological information collection device is in normal operation state, and the patient is in normal condition. When an emergency sign is detected from the patient, the patient emergency indicator turns red indicating the patient is in an emergency, the comprehensive patient biological information collection device initiates an emergency call to the nearby emergency dispatch center to notify a live emergency responder with the patient's GPS location information and patient information including contact information of patient's relatives and friends on record, the live emergency responder connects to one or more patient's relatives and friends on record and a nearby medical facility to coordinate immediate medical assistance to the patient based on the patient information received and retrieved from the patient database, and the live emergency responder and the emergency response system server provide patient specific medical assistance instructions for the patient to follow including instructing the patient to take one or more patient specific emergency medicines stored in the emergency medicine storage of the comprehensive patient biological information collection device, and the patient continues to communicate with the live emergency responder and the one or more patient's relatives and friends on record until an ambulance from a nearby medical facility arrives.

In certain embodiments, at the emergency response system server, patient's current status can be displayed whenever needed or by query to indicate in real time at least followings: whether the comprehensive patient biological information collection device is in normal operation state or not; whether the patient is in normal condition or not; whether the patient is in emergency situation or not; and whether responders are in actions or not. Data related to the patient's current status, actions and communications of response team(s) and any other persons involved are recorded and stored in the server or cloud.

In certain embodiments, the emergency response system server includes: a server processor, a network interface controller, and a non-volatile memory. The server processor controls operations of the emergency response system. The network interface controller connects to the communication network through a firewall connector over a firewall. The non-volatile memory stores a server operating system, a network communication module, and an emergency response controller having a patient information storage module for accessing the patient database through a database interface, a communication control module for facilitating communication to the communication system through a communication control interface, a patient information processing module, and computer executable instructions. When executed by the server processor, the computer executable instructions perform one or more of following operations:
receiving, through a patient communication module of the comprehensive patient biological information collection device of the patient, patient biological information constantly monitored and collected by the set of patient biological information collection sensors of the comprehensive patient biological information collection device;
processing, by the patient information processing module, the patient biological information received;
comparing, by the patient information processing module, the patient biological information processed against base line patient biological information from the patient database;
initiating, by the emergency response system server, an emergency voice call to the comprehensive patient biological information collection device of the patient through the communication system, when at least one of patient biological information collection sensors detects certain type of biological information that exceeds a normal range of the set of baseline biological information stored in the patient database;
initiating, by the emergency response system server, an emergency call to the nearby emergency dispatch center to notify the live emergency responder with the patient's GPS location information and patient information including contact information of patient's relatives and friends on record;
connecting, through the communication control module, the comprehensive patient biological information collection device carried by the patient to the patient's relatives and friends on record and a nearby medical facility to coordinate immediate medical assistance to the patient; and
transmitting, through the communication control module, a set of patient specific medical assistance instructions through the communication control interface and the live emergency responder for the patient to follow including instructing the patient to take one or more patient specific emergency medicines stored in the emergency medicine storage of the comprehensive patient biological information collection device, and the patient continues to communicate with the live emergency responder and the one or more patient's relatives and friends on record until an ambulance from the nearby medical facility arrives.

In certain embodiments, each of the set of comprehensive patient biological information collection devices includes: a processor, a network interface controller, and a non-volatile memory. The processor controls operations of the comprehensive patient biological information collection device. The network interface controller facilitates the communication among the comprehensive patient biological information collection device, the emergency response system server and the communication system over the communication network. The non-volatile memory stores an operating system, a GPS module for detecting the GPS location of the patient carrying the comprehensive patient biological information collection device, and a comprehensive patient biological information collection device controller having the patient communication module, a comprehensive patient biological information collection module, a comprehensive patient biological information processing module, a comprehensive patient biological information storage module, and computer executable instructions.

In certain embodiments, when executed by the processor, the computer executable instructions perform one or more of following operations:
receiving, through the comprehensive patient biological information collection module over the patient communication module, patient biological information constantly monitored and collected by a set of patient biological information collection sensors;
processing, by the comprehensive patient biological information processing module, the patient biological information received from the set of patient biological information collection sensors;
storing, by the comprehensive patient biological information storage module, the processed patient biological information;
and updating, by the patient communication module, the patient database of the emergency response system.

In certain embodiments, the comprehensive patient biological information collection device further includes:
the emergency button for the patient to press to notify the emergency response system when an emergency occurs;
the patient emergency indicator, when the patient is in normal condition, the patient emergency indicator is lit in green, and when the patient is in an emergency condition, the patient emergency indicator turns in red;
a display screen for the patient to receive and display text messages, emails, emergency assistance messages, and to carry out video calls;
a power supply module for providing electrical power to the comprehensive patient biological information collection device;
the emergency medicine storage for storing one or more patient specific emergency medicines for the patient; and
a SIM card slot for installing a SIM card for enabling mobile communication.

In certain embodiments, the communication among the patient, the live emergency responder, the patient's relatives and friends on record and the nearby medical facility includes: mobile voice calls, mobile videotelephony calls, landline voice calls, videotelephony call over the Internet, text messages over mobile phones, text messages over a set of social media platforms, and videotelephony calls over the set of social media platforms.

In certain embodiments, the communication system includes:
a voice input module having a mobile voice input interface to receive mobile voice calls, and a landline voice input interface to receive landline voice calls;
a text input module having a social media text input interface to receive text messages through the set of social media platforms, and a text message input interface to receive text messages through mobile phones;
a video input module having a mobile video input interface to receive video calls over the mobile phones, and a social media video input interface to receive video calls through the set of social media platforms;
a voice output module having a mobile voice output interface to make mobile voice calls, and a landline voice output interface to make landline voice calls;
a text output module having a social media text output interface to transmit text messages through the set of social media platforms, and a text message output interface to transmit text messages through the mobile phones;
a video output module having a mobile video output interface to make video calls over the mobile phones, and a social media video output interface to make video calls through the set of social media platforms;
a speech to text conversion module for converting voice input to text input;
a live emergency responder interface module for the live emergency responder to receive and make conference calls among the patient, the live emergency responder, the patient's relatives and friends on record and the nearby medical facility through voice calls, text messages, and video calls; and
a text to speech conversion module to make voice calls to the patient through the comprehensive patient biological information collection device.

In certain embodiments, the communication control interface includes: a voice input terminal connected to the voice input module, a text input terminal connected to the text input module, a video input terminal connected to the video input module, a voice output terminal connected to the voice output module, a text output terminal connected to the text output module, and a video output terminal connected to the video output module.

In yet another aspect, the present disclosure relates to a method of using an emergency response system having a comprehensive patient biological information collection device. In certain embodiments, the method includes:
registering, by a set of patients, each of the set of patients, and a set of comprehensive patient biological information collection devices, one corresponding comprehensive patient biological information collection device for each patient, at an emergency response system server of the emergency response system, wherein patient information of each of the set of patients is stored in a patient database of the emergency response system, and the emergency response system provide immediate emergency assistance to the patient when the comprehensive patient biological information collection device of the patient detects one or more emergency signs from the patient;
constantly monitoring and collecting, by a set of patient biological information collection sensors of the comprehensive patient biological information collection device, patient biological information of each patient;
receiving, through a patient communication control module 10664 from the comprehensive patient biological information collection device, the patient biological information constantly monitored and collected by the set of patient biological information collection sensors of the comprehensive patient biological information collection device;
processing, through a patient information processing module of the emergency response system server of the emergency response system, the patient biological information received, and comparing the patient biological information received with a set of baseline biological information stored in the patient database;
initiating, by the emergency response system server, at least voice communication between the emergency response system server and the comprehensive patient biological information collection device of the patient through the communication system directly, when at least one of the patient biological information collection sensors detects certain type of biological information that exceeds a normal range of the set of baseline biological information stored in the patient database;
initiating, by the emergency response system server, an emergency call to a nearby emergency dispatch center to notify a live emergency responder with the patient's GPS location information and patient information including contact information of patient's relatives and friends on record;
connecting, by the live emergency responder, to one or more patient's relatives and friends on record and a nearby medical facility to coordinate immediate medical assistance to the patient based on the patient information received and retrieved from the patient database;
providing, by the live emergency responder and the emergency response system server, patient specific medical assistance instructions for the patient to follow including instructing the patient to take one or more patient specific emergency medicines stored in an emergency medicine storage of the comprehensive patient biological information collection device; and
communicating, by the patient through a communication system of the emergency response system, with the live emergency responder and the one or more patient's relatives and friends on record until an ambulance from a nearby medical facility arrives.

In certain embodiments, the emergency response system includes: the emergency response system server, a communication network, the communication system, the patient database, and the set of comprehensive patient biological information collection devices. Each patient carries one comprehensive patient biological information collection device. Each patient and a corresponding comprehensive patient biological information collection device are registered at the emergency response system server. The emergency response system provides immediate emergency assistance to the patient when the corresponding comprehensive patient biological information collection device of the patient detects one or more emergency signs from the patient.

In certain embodiments, the patient database is connected to and accessible by the emergency response system server. The patient database stores patient information of all patients. The patient information includes personal information, medical history, a set of baseline biological information, patient contact information, and contact information of relatives and friends and local medical facilities to be notified. The communication system is also connected to the emergency response system server. The communication system provides voice, text, and video communication over the communication network among the patients, one or more live emergency responders from the nearby emergency dispatch center, one or more patient's relatives and friends on record, and one or more nearby medical facilities to provide immediate emergency assistance to the patient when emergency occurs.

In certain embodiments, each comprehensive patient biological information collection device collects patients' biological information through the set of patient biological information collection sensors and the set of patient biological information collection sensors constantly monitors the biological information of the patient and transmits the patient biological information collected to the emergency response system server through the comprehensive patient biological information collection device. When at least one of the patient biological information collection sensors detects certain type of biological information that exceeds a normal range of the set of baseline biological information stored in the patient database, the emergency response system server initiates at least voice communication between the emergency response system server and the comprehensive patient biological information collection device of the patient through the communication system directly. Each comprehensive patient biological information collection device also includes the emergency medicine storage for storing one or more patient specific emergency medicines to be used when emergency occurs.

These and other aspects of the present disclosure will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the present disclosure, and features and benefits thereof, and together with the written description, serve to explain the principles of the present invention. Wherever possible, the same reference numbers are used throughout the drawings to refer to the same or like elements of an embodiment, and wherein:

FIG. 1 shows a block diagram of a comprehensive patient biological information collection device according to certain embodiments of the present disclosure;

FIG. 2 shows a block diagram of a patient biological information collection module of the comprehensive patient biological information collection device using a set of patient biological information collection sensors to collect patient's biological information according to certain embodiments of the present disclosure;

FIG. 3 illustrates an exemplary set of patient biological information collection sensors used to collect patient biological information according to certain embodiments of the present disclosure;

FIG. 4 shows an exemplary comprehensive patient biological information collection device according to certain embodiments of the present disclosure;

FIG. 5 shows an exemplary robot as a patient biological information collection sensor for collecting patient biological information according to certain embodiments of the present disclosure;

FIG. 6 illustrates a block diagram of an emergency response system using a set of comprehensive patient biological information collection devices according to certain embodiments of the present disclosure;

FIG. 7 illustrates a block diagram of an emergency response system server of the emergency response system according to certain embodiments of the present disclosure;

FIG. 8 illustrates a block diagram of a communication system of the emergency response system according to certain embodiments of the present disclosure; and

FIG. 9 shows a flow chart of a method of using the emergency response system using the set of patient biological information collection sensors according to certain embodiments of the present disclosure.

### DETAILED DESCRIPTION

The present disclosure is more particularly described in the following examples that are intended as illustrative only since numerous modifications and variations therein will be apparent to those skilled in the art. Various embodiments of the disclosure are now described in detail. Referring to the drawings, like numbers, if any, indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present disclosure. Additionally, some terms used in this specification are more specifically defined below.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the disclosure or of any exemplified term. Likewise, the disclosure is not limited to various embodiments given in this specification.

Unless otherwise defined, all technical and scientific terms used herein have the same meaning as commonly understood by one of ordinary skill in the art to which this disclosure pertains. In the case of conflict, the present document, including definitions will control.

As used herein, “around”, “about” or “approximately” shall generally mean within 20 percent, preferably within 10 percent, and more preferably within 5 percent of a
given value or range. Numerical quantities given herein are approximate, meaning that the term “around”, “about” or “approximately” can be inferred if not expressly stated.

As used herein, “plurality” means two or more.

As used herein, the terms “comprising,” “including,” “carrying,” “having,” “containing,” “involving,” and the like are to be understood to be open-ended, i.e., to mean including but not limited to.

As used herein, the phrase at least one of A, B, and C should be construed to mean a logical (A or B or C), using a non-exclusive logical OR. It should be understood that one or more steps within a method may be executed in different order (or concurrently) without altering the principles of the present disclosure.

As used herein, the term module may refer to, be part of, or include an Application Specific Integrated Circuit (ASIC); an electronic circuit; a combinational logic circuit; a field programmable gate array (FPGA); a processor (shared, dedicated, or group) that executes code; other suitable hardware components that provide the described functionality; or a combination of some or all of the above, such as in a system-on-chip. The term module may include memory (shared, dedicated, or group) that stores code executed by the processor.

The term code, as used above, may include software, firmware, and/or microcode, and may refer to programs, routines, functions, classes, and/or objects. The term shared, as used above, means that some or all code from multiple modules may be executed using a single (shared) processor. In addition, some or all code from multiple modules may be stored by a single (shared) memory. The term group, as used above, means that some or all code from a single module may be executed using a group of processors. In addition, some or all code from a single module may be stored using a group of memories.

The apparatuses and methods described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

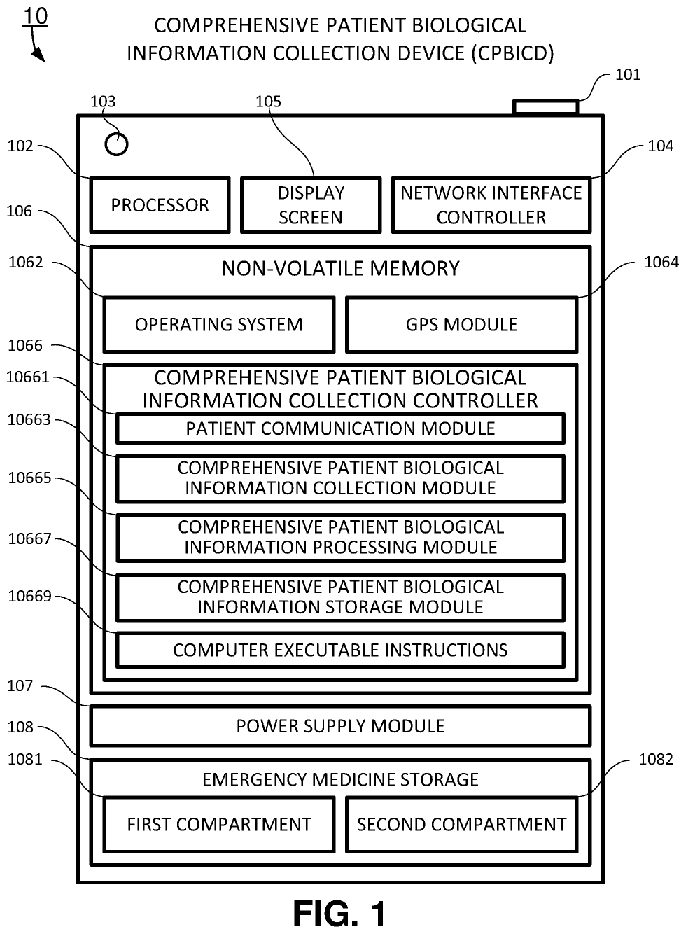

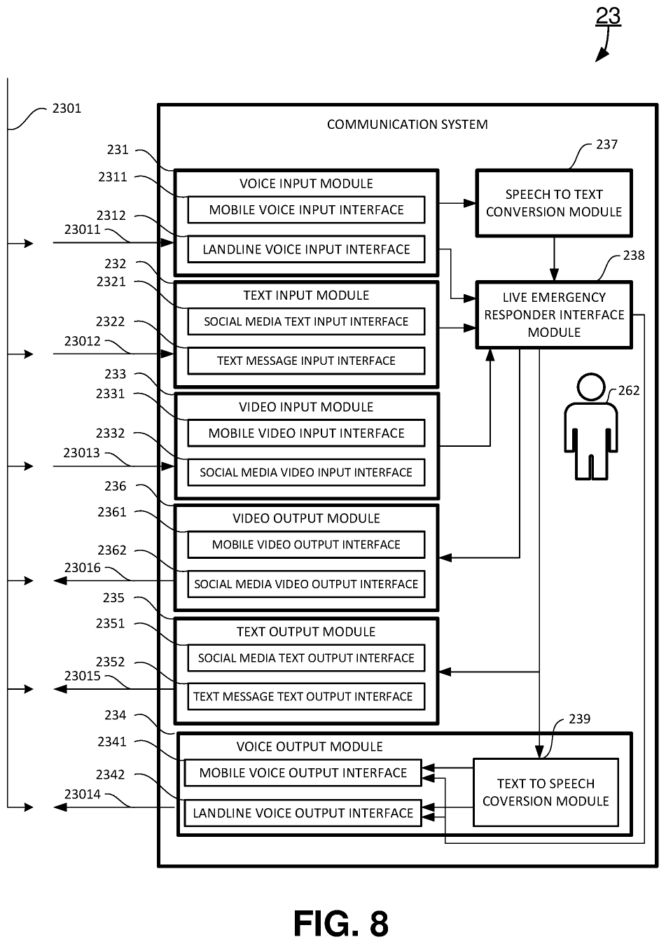

The present disclosure will now be described more fully hereinafter with reference to the accompanying drawings FIGS. 1 through 9, in which embodiments of the disclosure are shown. This disclosure may, however, be embodied in many different forms and should not be construed as limited to the embodiments set forth herein; rather, these embodiments are provided so that this disclosure will be thorough and complete, and will fully convey the scope of the disclosure to those skilled in the art. Like numbers refer to like elements throughout.

Currently, there is no Emergency Signs Early Detection, Alert and Response System in present market. To detect the early warning signs of emergency conditions, such as strokes and heart attacks, several vital signs must be measured or monitored, including, blood pressure, heart beat rate or pulse, body temperature, respiratory rate, blood sugar, and EKG. Medical devices are widely available for home use and affordable for ordinary families to collect the key biological or vital information as described above. However, there are several major challenges or deficiencies to use multiple standalone medical devices to collect critical biological information: 1) lack of a comprehensive software system that makes the collections, transmission, storage, and analysis of the data collected by standalone devices reliable and useful during the emergency situations; 2) it is not user-friendly to use multiple standalone medical devices to collect, transmit, store and use the data; 3) costs are much higher to manufacture standalone medical devices with their own hardware and software for the data collection, procession and analysis than a comprehensive one; and 4) waste of resources: while a comprehensive patient biological informational collection device can share the same hardware and software for the data processions, transmission, storage, and analysis, which could not only significantly reduce the waste of resources and costs of manufacture, but also become more user-friendlier. It is desirable to have a user-friendly, low-cost, and resource-saving alternative to monitor patients' biological information from multiple sensors in one comprehensive system and make the data collection much easier by patients themselves or any non-healthcare professional users and the information collected more reliable and useful.

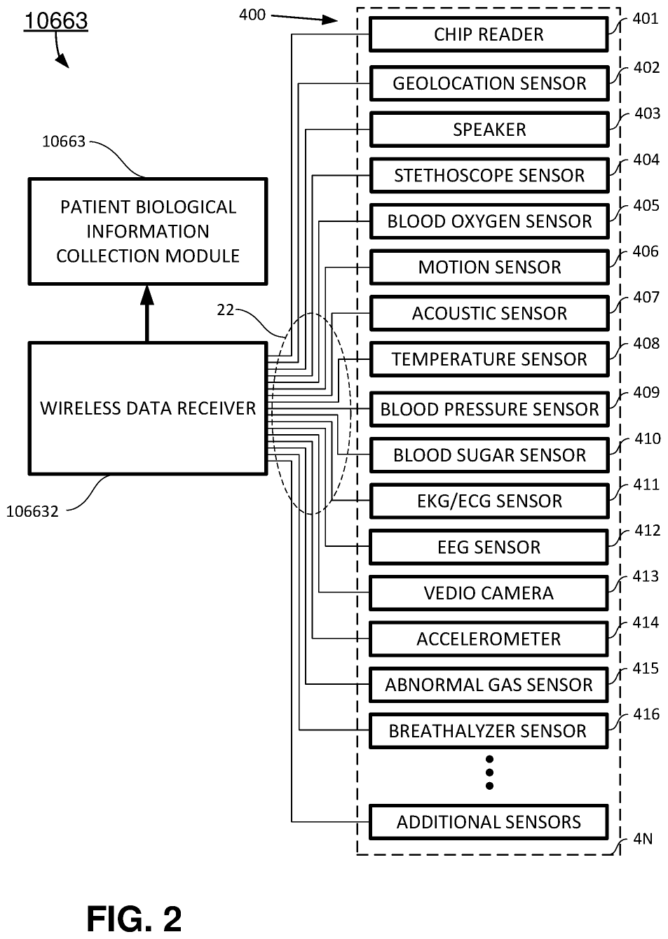

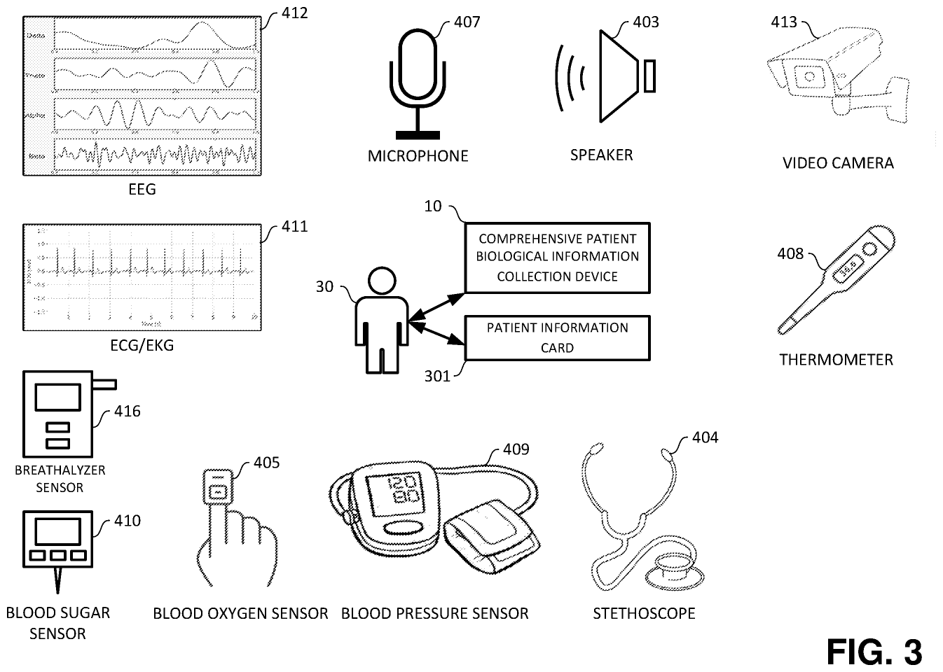

In one aspect, the present disclosure relates to a comprehensive patient biological information collection device 10, as shown in FIGS. 1-5. A group of the comprehensive patient biological information collection devices 10 is used by a group of patients 30, each patient 30 carries one comprehensive patient biological information collection device 10. In certain embodiments, the comprehensive patient biological information collection device 10, as shown in FIG. 1, collects biological information of a patient 30 through a set of patient biological information collection sensors 400, as shown in FIG. 2 and FIG. 3, and monitors the wellbeing of the patient 30. The comprehensive patient biological information collection device 10 includes: a processor 102, a network interface controller 104, and a non-volatile memory 106. The processor 102 controls operations of the comprehensive patient biological information collection device 10. The network interface controller 104 facilitates the communication among the comprehensive patient biological information collection device 10, and the set of patient biological information collection sensors 400 over a communication network 22.

In certain embodiments, the non-volatile memory 106 stores an operating system 1062, a GPS module 1064 for detecting the GPS location of the patient 30 carrying the comprehensive patient biological information collection device 10, and a comprehensive patient biological information collection device controller 1066 having a patient communication module 10661, a comprehensive patient biological information collection module 10663, a comprehensive patient biological information processing module 10665, a comprehensive patient biological information storage module 10667; and computer executable instructions 10669. The set of patient biological information collection sensors 400 collects patient's biological information through the comprehensive patient biological information collection module 10663, and processes the patient's biological information through the comprehensive patient biological information processing module 10665, in such a way that costs of monitoring patient's biological information is dramatically reduced because all processing of the data collected by the set of patient biological information collection sensors 400 is collectively processed through the processor 102 and the comprehensive patient biological information processing module 10665, instead of processed by each and every individual biological information monitoring machines/devices.

In certain embodiments, when executed by the processor 102, the computer executable instructions 10669 perform one or more of following operations:
receiving, through the comprehensive patient biological information collection module 10663 over the patient communication module 10661, patient biological information constantly monitored and collected by a set of patient biological information collection sensors 400;
processing, by the comprehensive patient biological information processing module 10665, the patient biological information received from the set of patient biological information collection sensors 400;
storing, by the comprehensive patient biological information storage module 10667, the processed patient biological information; and
updating, by the patient communication module 10661, a patient database 24.

In certain embodiments, the patient communication module 10661 facilitates communication among the patient database 24, the comprehensive patient biological information collection device controller 1066, and the set of patient biological information collection sensors 400 through the network interface controller 104 over the communication network 22. As shown in FIG. 2, the comprehensive patient biological information collection module 10663 collects patient biological information through a wireless data receiver 106632 over the communication network 22 from the set of patient biological information collection sensors 400 from the patient 30. The comprehensive patient biological information processing module 10665 processes the patient biological information collected through the comprehensive patient biological information collection module 10663. The comprehensive patient biological information storage module 10667 stores the patient biological information processed through the comprehensive patient biological information processing module 10665 and synchronizes the patient biological information stored with the patient database 24.

In certain embodiments, the communication network 22 includes a wireless personal area network (WPAN) having a Wi-Fi network, a Bluetooth network, an infrared network, and a Zigbee network, a wireless local area network (WLAN), a wireless metropolitan area network (WMAN), a wireless wide area network (WWAN), a cellular network, and a mobile communication network.

In certain embodiments, the set of patient biological information collection sensors 400 includes one or more of: a chip reader 401, a geolocation sensor 402, a speaker 403, a stethoscope sensor 404, a blood oxygen sensor 405, a motion sensor 406, an acoustic sensor 407, a temperature sensor 408, a blood pressure sensor 409, a blood sugar sensor 410, an EKG/ECG sensor 411, an EEG sensor 412, a video camera 413, an accelerometer 414, an abnormal gas sensor 415, and a breathalyzer sensor 416.

In certain embodiments, patient information can be stored in a credit card sized patient information card 301, as shown in FIG. 3. The patient information includes personal information, medical history, a set of baseline biological information, patient contact information, and contact information of relatives, friends and other responders and local medical facilities to be notified of each registered patient 30. When setting up a comprehensive patient biological information collection device 10 for a patient 30, the chip reader 401 can be used to upload important patient information into the comprehensive patient biological information collection device 10 very quickly without typing errors.

In certain embodiments, the geolocation sensor 402 is used to monitor the geolocation of the patient 30. Such geolocation sensor 402 can be a part of the comprehensive patient biological information collection device 10, an electronic watch, or a wearable device wirelessly connected to the comprehensive patient biological information collection device 10. It tracks the movement of the patient 30. If the patient gets into certain predetermined areas, it may trigger an emergency condition that certain people in his/her contact list may be notified.

In certain embodiments, the speaker 403, as well as an earphone, is used for the patient 30 to make and receive voice calls, and to provide voice instructions to the patient 30.

In certain embodiments, the stethoscope sensor 404 may be a separate device and will be used only when requested by a medical professional. When a remote medical professional or a live emergency responder 262 needs to check and monitor patient's heart, lung and breathing conditions, the patient 30 may be instructed to place the stethoscope sensor 404 in designated area of interest for the examination and monitoring.

In certain embodiments, as shown in FIG. 3, the stethoscope sensor 414 is used for monitoring breathing, heart, murmurs, Atrial Fibrillation (AFib), thoracic, arterial, intravenous, uterine, fetal, intestinal and other sounds of the patient 30. The stethoscope sensor 414 collects and monitors breathing, heart, thoracic, arterial, intravenous, uterine, fetal, intestinal and other sounds of the patient 30, then various sound samples collected are transmitted to the comprehensive patient biological information collection module 10663 and the comprehensive patient biological information processing module 10665 of the comprehensive patient biological information collection device 10 for sound signal processing using artificial intelligence algorithms. When the various sound samples received and analyzed deviate substantially from the normal baseline corresponding sound samples, the comprehensive patient biological information collection device 10 determines that the patient 30 is near or in an emergency condition, and the live emergency responder 262 or medical professionals may be notified.

In certain embodiments, the blood oxygen sensor 405 is used for monitoring patient's blood oxygen level. The blood oxygen sensor 405 may not connected to the patient 30 constantly, and may be used when directed by a medical professional. The blood oxygen sensor 405 may be used on a daily basis to monitor the patient's blood oxygen level.

In certain embodiments, the motion sensor 406 is widely used for monitoring patient's movement. Such the motion sensor 406 can be a part of the comprehensive patient biological information collection device 10, an electronic watch, or a wearable device wirelessly connected to the comprehensive patient biological information collection device 10. When the patient 30 moves in an unusual pace, speed, or sometimes combined with the accelerometer 414 when an impact to the patient 30 is detected, an emergency condition may be triggered so that certain people in his/her contact list may be notified.

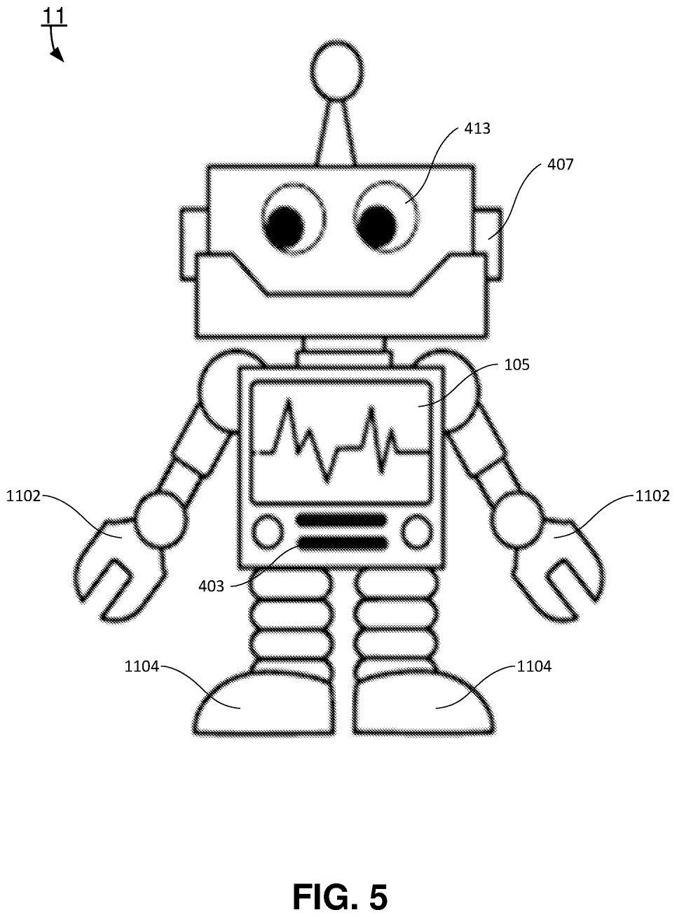

In certain embodiments, the acoustic sensor 407, or a microphone, is used to make and receive voice calls by the patient 30. The acoustic sensor 407 can be installed in the comprehensive patient biological information collection device 10, or a robot 11, as shown in FIG. 5. The acoustic sensor 407 may be used to generate voice samples of the patient 30 for diagnose purposes. When the patient 30 speaks at an unusual pace, unclear, or sluggish, it may trigger an emergency condition that certain people in his/her contact list may be notified. In certain embodiments, the acoustic sensor 407 may be used by the live emergency responder 262 or medical professionals to detect background sounds surrounding the patient 30.

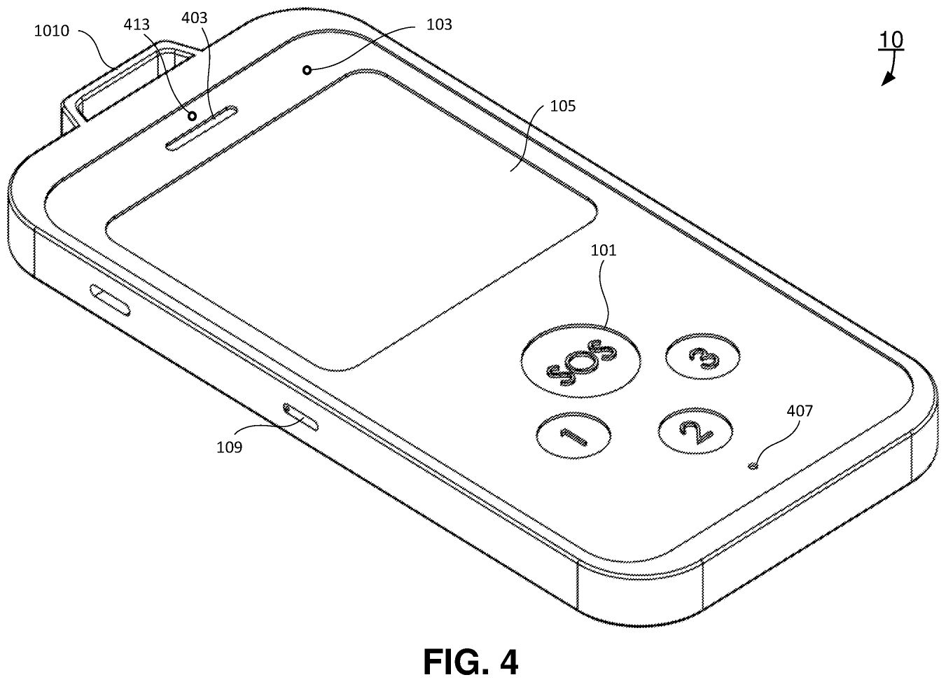

In certain embodiments, as shown in FIGS. 4 and 5, the acoustic sensor 407 (or a microphone) and the speaker 403 are used to allow the patient 30 to make and receive voice calls, to generate voice samples of the patient 30, and to provide voice instructions to the patient 30. The acoustic sensor 407 may also be used to collect speech samples of the patient 30. When the patient 30 is experiencing difficulty speaking, the speech samples collected will deviate from the normal speech patterns, for example, the tempo of the speech, the pitch, the intensity may change dramatically. These deviations may be picked up through the comprehensive patient biological information collection module 10663 and the comprehensive patient biological information processing module 10665 of the comprehensive patient biological information collection device 10 through speech processing using artificial intelligence algorithms. When these speech parameters such as the tempo, the pitch, and the intensity are determined to exceed a normal range of these speech parameters, the comprehensive patient biological information collection device 10 determines that the patient 30 is near or in an emergency condition, and the live emergency responder 262 or medical professionals may be notified.

In certain embodiments, the voice data from the user is used as user's identifier the same way as the fingerprinting as the fingerprinting to determine the user's identity.

In certain embodiments, the temperature sensor 408 is used for monitoring patient's body temperature. Such the temperature sensor 408 can be a part of the comprehensive patient biological information collection device 10, an electronic watch, or a wearable device wirelessly connected to the comprehensive patient biological information collection device 10. One or more temperature sensors may be placed in predetermined locations of the patient 30. These sensors collect and monitor body temperatures of the patient 30 in predetermined intervals. The temperatures collected are transmitted to the comprehensive patient biological information collection module 10663 and the comprehensive patient biological information processing module 10665 of the comprehensive patient biological information collection device 10 for temperature processing using artificial intelligence algorithms. When the temperatures received and analyzed deviate substantially from the normal baseline corresponding temperature data, or body temperature of the patient 30 exhibits sudden change, the comprehensive patient biological information collection device 10 determines that the patient 30 is near or in an emergency condition and the live emergency responder 262 or medical professionals may be notified.

In certain embodiments, the blood pressure sensor 409 is used for monitoring patient's blood pressure and pulse rate. Such the blood pressure sensor 409 can be a part of the comprehensive patient biological information collection device 10, an electronic watch, or a wearable device wirelessly connected to the comprehensive patient biological information collection device 10. The blood pressure sensor 409 can ensure the heart and blood vessel's health of the patient 30 and detect emergency signs before a stroke or heart attack happens. When blood pressure of the patient 30 exceeds certain normal range, an emergency condition may be triggered and the live emergency responder 262 or medical professionals may be notified.

In certain embodiments, the blood sugar sensor 410 is used for monitoring patient's blood sugar level. Such the blood sugar sensor 410 can be a part of the comprehensive patient biological information collection device 10, an electronic watch, or a wearable device wirelessly connected to the comprehensive patient biological information collection device 10. The blood sugar sensor 410 is very important electronic equipment for diabetic patients. When blood sugar level of the patient 30 exceeds certain normal range, an emergency condition may be triggered and the live emergency responder 262 or medical professionals may be notified.

In certain embodiments, the EKG/ECG sensor 411 is used for monitoring patient's electrocardiograms. Such EKG/ECG sensor 411 can be a part of the comprehensive patient biological information collection device 10, an electronic watch, or a wearable device wirelessly connected to the comprehensive patient biological information collection device 10. When electrocardiograms patterns of the patient 30 indicate any abnormality, an emergency condition may be triggered and the live emergency responder 262 or medical professionals may be notified.

In certain embodiments, as shown in FIG. 3, the electrocardiograms 1405 are used for monitoring electrocardiography of the patient 30. Full featured electrocardiograms 1405 may be found from doctor's offices, however, basic electrocardiograms 1405 may be found in wearable mobile devices such as Apple Watches. The patient 30 can use the full-featured electrocardiograms 1405 occasionally in doctor's office, a smart phone-based electrocardiograms 1405 may be used to monitor electrocardiography data in a continuous manner. The Apple Watch includes sensors to collect and monitor the electrocardiography data of the patient 30, then the electrocardiography data collected is wirelessly transmitted to the comprehensive patient biological information collection device 10. The electrocardiography data collected is transmitted and processed at the comprehensive patient biological information collection module 10663 and the comprehensive patient biological information processing module 10665 of the comprehensive patient biological information collection device 10 for electrocardiography data processing using artificial intelligence algorithms. When the electrocardiography data received and analyzed deviates substantially from the normal baseline electrocardiography data, the comprehensive patient biological information collection device 10 determines that the patient 30 is near or in an emergency condition, and the live emergency responder 262 or medical professionals may be notified.

In certain embodiments, the EEG sensor 412 is used for monitoring patient's electroencephalograms. Such EEG sensor 412 can be a part of the comprehensive patient biological information collection device 10, an electronic watch, or a wearable device wirelessly connected to the comprehensive patient biological information collection device 10. When electroencephalograms patterns of the patient 30 indicate any abnormality, an emergency condition may be triggered and the live emergency responder 262 or medical professionals may be notified.

In certain embodiments, as shown in FIG. 3, the electroencephalograms are used for monitoring electroencephalography of the patient 30. Due to the expensive nature of the electroencephalograms, the patient 30 may not be able to afford to have an electroencephalogram installed at home or office. However, the comprehensive patient biological information collection device 10 may include wireless interfaces to the electroencephalogram installed in doctor's office, and the patient 30 may be able to go to doctor's office regularly and obtain electroencephalography of the patient 30. The comprehensive patient biological information collection device 10 receives electroencephalography data and transmits the electroencephalography data received to the comprehensive patient biological information collection module 10663 and the comprehensive patient biological information processing module 10665 of the comprehensive patient biological information collection device 10 for electroencephalographical data processing using artificial intelligence algorithms. When the electroencephalographical data received and analyzed deviates substantially from the normal baseline electroencephalographical data, the comprehensive patient biological information collection device 10 determines that the patient 30 is near or in an emergency condition, and the live emergency responder 262 or medical professionals may be notified.

In certain embodiments, the video camera 413 is used for monitoring patient's posture, walking patterns and movement. The video camera 413 can be installed in a home, on the comprehensive patient biological information collection device 10, as shown in FIG. 4, or on a robot 11, as shown in FIG. 5. The video camera 413 also allows the patient 20 to conduct video conference with caretakers, the live emergency responder 262 or medical professionals. When an unusual walking pattern, or unusual posture is detected by the video camera 413, an emergency condition may be triggered and the live emergency responder 262 or medical professionals may be notified.

In certain embodiments, as shown in FIG. 4 and FIG. 5, the video camera 413 may be used with a display screen 105 to receive and display text messages and carry out video calls. Additionally, the video camera 413 are also used to monitoring the patient through videos and still images. In one embodiment, a video camera 413 is equipped on the comprehensive patient biological information collection device 10, as shown in FIG. 4, or on a robot 11. In another embodiments, a video camera 413 is installed on the wall of a room the patient stays in most of times, such as a room in a house, or room in an office. The wall-mount video camera 413 may be wirelessly connected to the comprehensive patient biological information collection device 10. The video camera 413 are used to collect videos and still images of the patient 30 during his/her daily life. The baseline biological information of the patient 30 includes normal facial images and normal walking videos of the patient 30 from many different angles and directions. Pre-stroke patient may experience certain facial expression changes such as facial paralysis, and walking patterns may also change. These deviations may be picked up through the comprehensive patient biological information collection module 10663 and the comprehensive patient biological information processing module 10665 of the comprehensive patient biological information collection device 10 through image and video processing using artificial intelligence algorithms. When these facial expressions and walking patterns of the patient 30 are determined to exceed a normal range of baseline images or baseline videos, the comprehensive patient biological information collection device 10 determines that the patient 30 is near or in an emergency condition, and the live emergency responder 262 or medical professionals may be notified.

In certain embodiments, the accelerometer 414 is used for detecting impact to the patient 30 and sudden fall of the patient 30. Such accelerometer 414 can be a part of the comprehensive patient biological information collection device 10, an electronic watch, or a wearable device wirelessly connected to the comprehensive patient biological information collection device 10. When the accelerometer 414 detects certain impact to the patient 30, or sudden fall of the patient 30, an emergency condition may be triggered and the live emergency responder 262 or medical professionals may be notified.

In certain embodiments, the abnormal gas sensor 415 is not necessarily a part of patient's biological information but it is related to the wellbeing of the patient 30. The abnormal gas sensor 415 is used for detecting harmful gas around the patient 30. When harmful gas such as natural gas, gasoline vapor, carbon monoxide, carbon dioxide is detected in higher than normal concentration around the patient 30, an emergency condition may be triggered and the live emergency responder 262 or medical professionals may be notified.

In certain embodiments, the breathalyzer sensor 416 is used for detecting blood alcohol level of the patient 30. The breathalyzer sensor 416 can be installed in the comprehensive patient biological information collection device 10 carried by the patient or in a car driven by the patient 30, or a wearable device wirelessly connected to the comprehensive patient biological information collection device 10. When the patient 30 is detected to have unusual high blood alcohol level, the comprehensive patient biological information collection device 10 may disable the car driven by the patient 30 wirelessly, and an emergency condition may be triggered and the live emergency responder 262 or medical professionals may be notified.

In certain embodiments, as shown in FIG. 3, the breathalyzer sensor 416 is used to analyze breath samples of the patient 30. The breathalyzer sensor 416 tests many elements such as blood alcohol level, nicotine, hard to digest food components such as lactose, fructose, sorbitol, and inulin to monitor the overall health of the patient 30. The breathalyzer sensor 416 can be made in a small portable package and wirelessly connected to the comprehensive patient biological information collection device 10 through home network, Wi-Fi, and Bluetooth. The results of analyzed breath samples are transmitted to the comprehensive patient biological information collection module 10663 and the comprehensive patient biological information processing module 10665 of the comprehensive patient biological information collection device 10 for breath sample data processing using artificial intelligence algorithms. When the analyzed breath sample data received and analyzed deviates substantially from the normal baseline breath sample data, the comprehensive patient biological information collection device 10 determines that the patient 30 is near or in an emergency condition, and the live emergency responder 262 or medical professionals may be notified.

In one embodiment, as shown in FIG. 5, the comprehensive patient biological information collection device 10 is a robot 11. The robot 11 has at least one pair of robotic arms 1102 to perform certain medical assistance such as carrying out certain medical tests. The robot 11 also has at least one pair of robotic feet 1104 to follow the patient 30 around his/her home and/or office, and monitor the patient 30's biological information. The robot 11 has eyes (video cameras 413), ears (microphone 407) and mouth (speakers 403) to monitor the patient facial expression, walking, and speaking. Following patient behaviors may be considered abnormal, and it is worth further investigation: (1) when the facial expressions become very animated and unsymmetrically, (2) when the patient drags his feet, walking much slower than usual, or having difficulty walking, (3) when the patient having difficulty speaking clearly, talking much slower than usual, having difficulty choosing the words, and having difficulty carrying out normal conversation. Under these circumstances, the robot 11 may offer some assistance to measure the blood pressure, heart beat rate, blood sugar level, blood oxygen level using various sensors over the robot 11. After preliminary examinations, and when the robot 11 determines that the patient 30 is in danger of suffering severer medical issues, the robot 11 may contact the live emergency responder 262 or medical professionals for assistance.

In another embodiment, the comprehensive patient biological information collection device 10 is a portable device. The portable the comprehensive patient biological information collection device 10 is carried with the patient 30 and the portable comprehensive patient biological information collection device 10 also stores one or more patient specific emergency medicines. In yet another embodiment, the portable the comprehensive patient biological information collection device 10 is a stationary comprehensive patient biological information collection device. The stationary ESEDARS personal device is placed at home or work place of the patient and the stationary comprehensive patient biological information collection device also stores one or more patient specific emergency medicines. In yet another embodiment, the ESEDARS personal device can be a group of public stationary comprehensive patient biological information collection device. These public stationary comprehensive patient biological information collection devices are placed in public places and each of the public stationary comprehensive patient biological information collection devices stores at least one of several common emergency medicines.

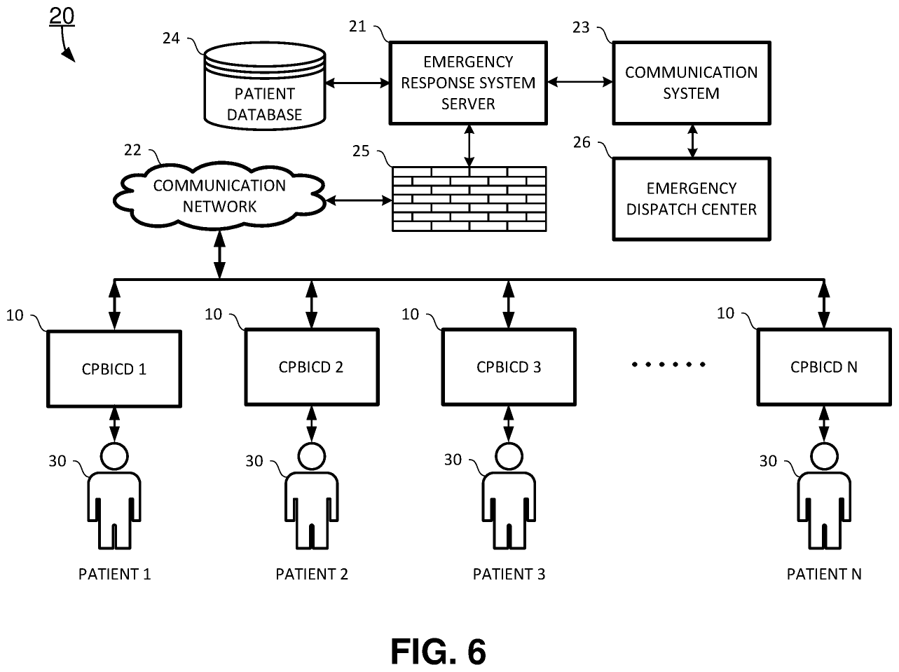

In another aspect, the present disclosure relates to an emergency response system as shown in FIG. 6-FIG. 9. In one embodiment, the emergency response system 20 can be triggered either by a patient pressing an emergency button 101 when a patient 30 realizes that he/she may be in an emergency situation, and the patient 30 is still conscious and able to press the emergency button 101. In another embodiment, when the patient 30 fell unconscious, and unable to press the emergency button 101, a comprehensive patient biological information collection device 10 carries by the patient 30 may trigger the emergency response system 20. The comprehensive patient biological information collection device 10 constantly monitors certain patient biological information, and triggers emergency response system 20 when the comprehensive patient biological information collection device 10 detects certain type of biological information that exceeds a normal range of the set of baseline biological information stored in a patient database 24.

In certain embodiments, the emergency response system can also be triggered remotely either by one or more live emergency responders 262 or a healthcare provider who is registered in the system and with a specialized App of this system installed in any of his/her smart devices, such as a smart phone, to collect the biological or medical information from, to give medical advices given to, and establish communication channels with the patient in need for help who carries or has access to the comprehensive patient biological information collection device.

In certain embodiments, as shown in FIG. 6, the emergency response system 20 includes: an emergency response system server 21, a communication network 22, a communication system 23, the patient database 24, and a set of comprehensive patient biological information collection devices 10. Each patient 30 carries one comprehensive patient biological information collection device 10. Each patient 30 and a corresponding comprehensive patient biological information collection device 10 are registered at the emergency response system server 21. The emergency response system 20 provides immediate emergency assistance to the patient 30 when the corresponding comprehensive patient biological information collection device 10 of the patient 30 detects one or more emergency signs from the patient 30.

In certain embodiments, the patient database 24 is connected to and accessible by the emergency response system server 21. The patient database 24 stores patient information of all patients 30. The patient information includes personal information, medical history, a set of baseline biological information, patient contact information, and contact information of relatives and friends and local medical facilities to be notified. The communication system 23 is also connected to the emergency response system server 21. The communication system 23 provides voice, text, and video communication over the communication network 22 among the patients 30, one or more live emergency responders 262 from a nearby emergency dispatch center 26, one or more patient's relatives and friends on record, and one or more nearby medical facilities to provide immediate emergency assistance to the patient when emergency occurs.

In certain embodiments, each comprehensive patient biological information collection device 10 collects patients' biological information through a set of patient biological information collection sensors 400 and the set of patient biological information collection sensors 400 constantly monitors the biological information of the patient 30 and transmits the patient biological information collected to the emergency response system server 21 through the comprehensive patient biological information collection device 10. When at least one of the patient biological information collection sensors 400 detects certain type of biological information that exceeds a normal range of the set of baseline biological information stored in the patient database 24, the emergency response system server 21 initiates at least voice communication between the emergency response system server 21 and the comprehensive patient biological information collection device 10 of the patient 30 through the communication system 23 directly. Each comprehensive patient biological information collection device 10 also includes an emergency medicine storage 108 for storing one or more patient specific emergency medicines to be used when emergency occurs.

In certain embodiments, a patient emergency indicator 103 of the comprehensive patient biological information collection device 10 is lit in green light indicating the comprehensive patient biological information collection device 10 is in normal operation state, and the patient 30 is in normal condition. When an emergency sign is detected from the patient 30, the patient emergency indicator 103 turns red indicating the patient is in an emergency, the comprehensive patient biological information collection device 10 initiates an emergency call to the nearby emergency dispatch center 26 to notify a live emergency responder 262 with the patient's GPS location information and patient information including contact information of patient's relatives and friends on record, the live emergency responder 262 connects to one or more patient's relatives and friends on record and a nearby medical facility to coordinate immediate medical assistance to the patient 30 based on the patient information received and retrieved from the patient database 24, and the live emergency responder 262 and the emergency response system server 21 provide patient specific medical assistance instructions for the patient 30 to follow including instructing the patient 30 to take one or more patient specific emergency medicines stored in the emergency medicine storage 108 of the comprehensive patient biological information collection device 10, and the patient 30 continues to communicate with the live emergency responder 262 and the one or more patient's relatives and friends on record until an ambulance from a nearby medical facility arrives.

In certain embodiments, the communication network 22 includes a wireless personal area network (WPAN) having a Wi-Fi network, a Bluetooth network, an infrared network, and a Zigbee network, a wireless local area network (WLAN), a wireless metropolitan area network (WMAN), a wireless wide area network (WWAN), a cellular network, and a mobile communication network.

In certain embodiments, at the emergency response system server 21, patient's current status can be displayed whenever needed or by query to indicate in real time at least followings: whether the comprehensive patient biological information collection device is in normal operation state or not; whether the patient is in normal condition or not; whether the patient is in emergency situation or not; and whether responders are in actions or not. Data related to the patient's current status, actions and communications of response team(s) and any other persons involved are recorded and stored in the server or cloud.

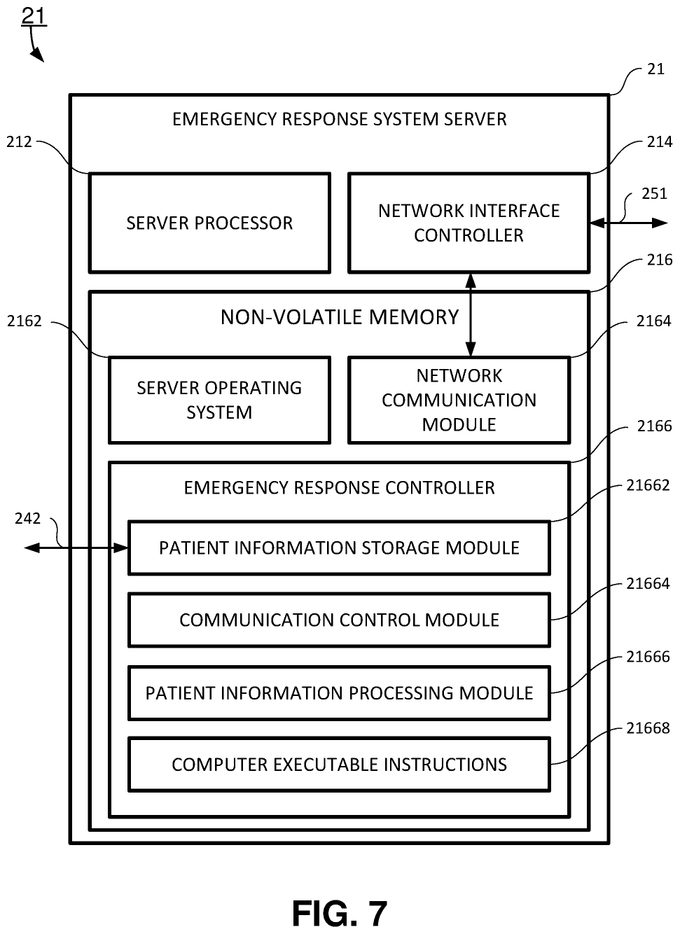

In certain embodiments, as shown in FIG. 7, the emergency response system server 21 includes: a server processor 212, a network interface controller 214, and a non-volatile memory 216. The server processor 212 controls operations of the emergency response system 20. The network interface controller 214 connects to the communication network 22 through a firewall connector 251 over a firewall 25. The non-volatile memory 216 stores a server operating system 2162, a network communication module 2164, and an emergency response controller 2166 having a patient information storage module 21662 for accessing the patient database 24 through a database interface 242, a communication control module 21664 for facilitating communication to the communication system 23 through a communication control interface 2301, a patient information processing module 21666, and computer executable instructions 21668. When executed by the server processor 212, the computer executable instructions 21668 perform one or more of following operations:
receiving, through a patient communication module 10661 of the comprehensive patient biological information collection device 10 of the patient 30, patient biological information constantly monitored and collected by the set of patient biological information collection sensors 400 of the comprehensive patient biological information collection device 10;
processing, by the patient information processing module 21666, the patient biological information received;
comparing, by the patient information processing module 21666, the patient biological information processed against base line patient biological information from the patient database 24;
initiating, by the emergency response system server 21, an emergency voice call to the comprehensive patient biological information collection device 10 of the patient 30 through the communication system 23, when at least one of the patient biological information collection sensors 400 detects certain type of biological information that exceeds a normal range of the set of baseline biological information stored in the patient database 24;
initiating, by the emergency response system server 21, an emergency call to the nearby emergency dispatch center 26 to notify the live emergency responder 262 with the patient's GPS location information and patient information including contact information of patient's relatives and friends on record;
connecting, through the communication control module 21664, the comprehensive patient biological information collection device 10 carried by the patient 30 to the patient's relatives and friends on record and a nearby medical facility to coordinate immediate medical assistance to the patient 30; and
transmitting, through the communication control module 21664, a set of patient specific medical assistance instructions through the communication control interface 2301 and the live emergency responder 262 for the patient 30 to follow including instructing the patient to take one or more patient specific emergency medicines stored in the emergency medicine storage 108 of the comprehensive patient biological information collection device 10, and the patient 30 continues to communicate with the live emergency responder 262 and the one or more patient's relatives and friends on record until an ambulance from the nearby medical facility arrives.

In certain embodiments, as shown in FIG. 1, each comprehensive patient biological information collection device 10 includes: a processor 102, a network interface controller 104, and a non-volatile memory 106. The processor 102 controls operations of the comprehensive patient biological information collection device 10. The network interface controller 104 facilitates the communication among the comprehensive patient biological information collection device 10, the emergency response system server 21 and the communication system 23 over the communication network 22. The non-volatile memory 106 stores an operating system 1062, a GPS module 1064 for detecting the GPS location of the patient 30 carrying the comprehensive patient biological information collection device 10, and a comprehensive patient biological information collection device controller 1066 having the patient communication module 10661, a comprehensive patient biological information collection module 10663, a comprehensive patient biological information processing module 10665, a comprehensive patient biological information storage module 10667, and computer executable instructions 10669.

In certain embodiments, when executed by the processor 102, the computer executable instructions 10669 perform one or more of following operations:
receiving, through the comprehensive patient biological information collection module 10663 over the patient communication module 10661, patient biological information constantly monitored and collected by a set of patient biological information collection sensors 400;
processing, by the comprehensive patient biological information processing module 10665, the patient biological information received from the set of patient biological information collection sensors 400;
storing, by the comprehensive patient biological information storage module 10667, the processed patient biological information;
and updating, by the patient communication module 10661, the patient database 24 of the emergency response system 20.

In certain embodiments, the patient biological information includes: speech samples, facial images and video, electroencephalography (EEG) data, electrocardiography (ECG/EKG) data, breath samples, blood glucose content, blood oxygen content, pulse rate, blood pressure, sound samples such as breathing, heart, thoracic, arterial, intravenous, uterine, fetal, intestinal and other sounds, and body temperatures at various time. The baseline biological information stored in the patient database 24 may include: normal speech samples including tempo, pitch, and intensity of speech, normal facial images and normal walking videos of the patient 30 from many different angles and directions, normal electroencephalography (EEG) data, normal electrocardiography (ECG/EKG) data, normal breath samples, normal blood glucose content range, normal blood oxygen content range, normal pulse rate range, normal blood pressure range, normal sound samples such as breathing, heart, thoracic, arterial, intravenous, uterine, fetal, intestinal and other sounds, and normal body temperatures range at various time.

In certain embodiments, the set of patient biological information collection sensors 400 includes one or more of:
a chip reader 401 for reading a patient information card 301 where the patient information is stored;
a geolocation sensor 402 for monitoring the geolocation of the patient 30;
a speaker 403 for the patient 30 to make and receive voice calls, and to provide voice instructions to the patient 30;
a stethoscope sensor 404 for monitoring patient's heart, lung and breathing conditions, including breathing, heartbeat, thoracic, arterial, intravenous, uterine, fetal, intestinal and other sounds at various time;
a blood oxygen sensor 405 for monitoring patient's blood oxygen level;
a motion sensor 406 for monitoring patient's movement;
an acoustic sensor 407 for making and receiving voice calls by the patient 30, generating voice samples of the patient 30, and detecting background sounds surrounding the patient 30;
a temperature sensor 408 for monitoring patient's body temperature;
a blood pressure sensor 409 for monitoring patient's blood pressure;
a blood sugar sensor 410 for monitoring patient's blood sugar level;
an EKG/ECG sensor 411 for monitoring patient's electrocardiograms;
an EEG sensor 412 for monitoring patient's electroencephalograms;
a video camera 413 for monitoring patient's posture, movement, facial and emotional expressions;
an accelerometer 414 for detecting impact to the patient 30 and sudden fall of the patient 30;
an abnormal gas sensor 415 for detecting harmful gas around the patient 30; and
a breathalyzer sensor 416 for detecting blood alcohol level of the patient 30.

In certain embodiments, as shown in FIG. 1, the comprehensive patient biological information collection device 10 further includes:
the emergency button 101 for the patient 30 to press to notify the emergency response system 20 when an emergency occurs;
the patient emergency indicator 103, when the patient 30 is in normal condition, the patient emergency indicator 103 is lit in green, and when the patient 30 is in an emergency condition, the patient emergency indicator 103 turns in red;
a display screen 105 for the patient 30 to receive and display text messages, emails, emergency assistance messages, and to carry out video calls;
a power supply module 107 for providing electrical power to the comprehensive patient biological information collection device 10;
the emergency medicine storage 108 for storing one or more patient specific emergency medicines for the patient 30; and
a SIM card slot 109 for installing a SIM card for enabling mobile communication.

In one embodiment, the emergency medicine storage 108 includes a first emergency medicine storage 1081, and a second emergency medicine storage 1082, as shown in FIG. 1. The first emergency medicine storage 1081 and the second emergency medicine storage 1082 store one or more patient specific emergency medicines to be used by the patient 30 carrying the comprehensive patient biological information collection device 10 when emergency occurs. In one embodiment, the patient specific emergency medicines include tissue plasminogen activator (tPA) for stroke/heart attack patients. In another embodiment, the patient specific emergency medicines include epinephrine for server allergic reaction patients. Various sizes of emergency medicine storage 108 of the comprehensive patient biological information collection device 10 may be available to accommodate various size emergency medicines for various high risk patients 30 with various known diseases.

In certain embodiments, as shown in FIG. 4, the comprehensive patient biological information collection device 10 includes the SIM card holder 109 to hold a SIM card for mobile communication capable comprehensive patient biological information collection device 10.

In certain embodiments, the communication among the patient 30, the live emergency responder 262, the patient's relatives and friends on record and the nearby medical facility includes: mobile voice calls, mobile videotelephony calls, landline voice calls, videotelephony call over the Internet, text messages over mobile phones, text messages over a set of social media platforms, and videotelephony calls over the set of social media platforms.

In certain embodiments, the communication system 23 includes:
a voice input module 231 having a mobile voice input interface 2311 to receive mobile voice calls, and a landline voice input interface 2312 to receive landline voice calls;
a text input module 232 having a social media text input interface 2321 to receive text messages through the set of social media platforms, and a text message input interface 2322 to receive text messages through mobile phones;
a video input module 233 having a mobile video input interface 2331 to receive video calls over the mobile phones, and a social media video input interface 2332 to receive video calls through the set of social media platforms;
a voice output module 234 having a mobile voice output interface 2341 to make mobile voice calls, and a landline voice output interface 2342 to make landline voice calls;
a text output module 235 having a social media text output interface 2351 to transmit text messages through the set of social media platforms, and a text message output interface 2352 to transmit text messages through the mobile phones;
a video output module 236 having a mobile video output interface 2361 to make video calls over the mobile phones, and a social media video output interface 2362 to make video calls through the set of social media platforms;
a speech to text conversion module 237 for converting voice input to text input;
a live emergency responder interface module 238 for the live emergency responder 262 to receive and make conference calls among the patient 30, the live emergency responder 262, the patient's relatives and friends on record and the nearby medical facility through voice calls, text messages, and video calls; and
a text to speech conversion module 239 to make voice calls to the patient 30 through the comprehensive patient biological information collection device 10.

In certain embodiments, the social media platforms include, but not limited to: Facebook, YouTube, Telegram, Parler, WhatsApp, Messenger, WeChat, Instagram, QQ, Tumblr, Qzone, Tik Tok, Sina Weibo, Twitter, Reddit, Baidu Tieba, LinkedIn, Viber, Snapchat, and Pinterest and various combination of these social media platforms.

In certain embodiments, the communication control interface 2301 includes: a voice input terminal 23011 connected to the voice input module 231, a text input terminal 23012 connected to the text input module 232, a video input terminal 23013 connected to the video input module 233, a voice output terminal 23014 connected to the voice output module 234, a text output terminal 23015 connected to the text output module 235, and a video output terminal 23016 connected to the video output module 236.

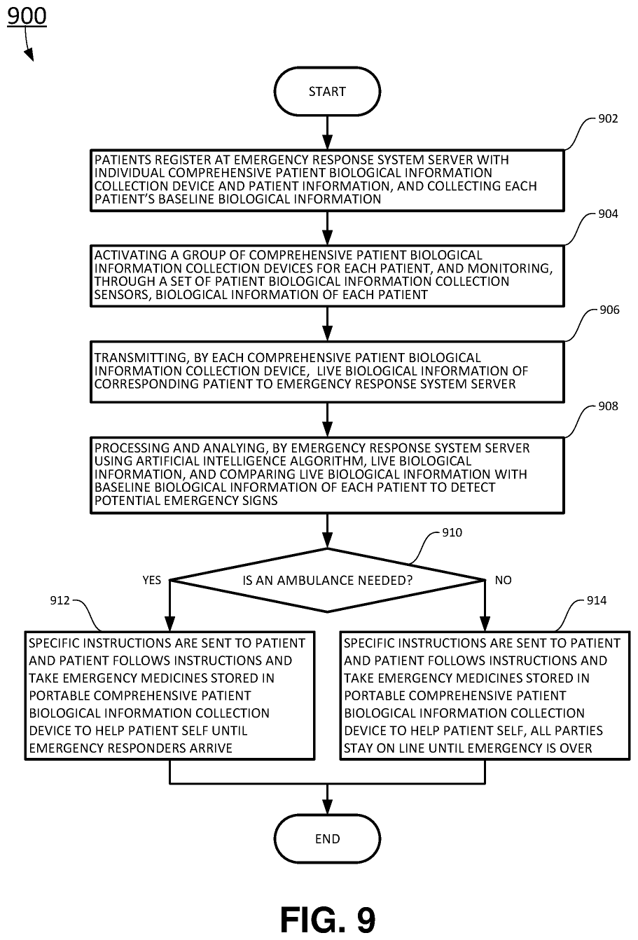

In yet another aspect, as shown in FIG. 9, the present disclosure relates to a method of using an emergency response system 20 having a set of comprehensive patient biological information collection devices 10. In certain embodiments, the method includes:

registering, by a set of patients 30, each of the set of patients 30, and a set of comprehensive patient biological information collection devices 10, one corresponding comprehensive patient biological information collection device 10 for each patient 30, at an emergency response system server 21 of the emergency response system 20, wherein patient information of each of the set of patients 30 is stored in a patient database 24 of the emergency response system 20, and the emergency response system 20 provide immediate emergency assistance to the patient 30 when the comprehensive patient biological information collection device 10 of the patient 30 detects one or more emergency signs from the patient 30;
constantly monitoring and collecting, by a set of patient biological information collection sensors 400 of the comprehensive patient biological information collection device 10, patient biological information of each patient 30;
receiving, through a patient communication control module 10664 from the comprehensive patient biological information collection device 10, the patient biological information constantly monitored and collected by the set of patient biological information collection sensors 400 of the comprehensive patient biological information collection device 10;
processing, through a patient information processing module 21666 of the emergency response system server 21 of the emergency response system 20, the patient biological information received, and comparing the patient biological information received with a set of baseline biological information stored in the patient database 24;
initiating, by the emergency response system server 21, at least voice communication between the emergency response system server 21 and the comprehensive patient biological information collection device 10 of the patient 30 through the communication system 23 directly, when at least one of the patient biological information collection sensors 400 detects certain type of biological information that exceeds a normal range of the set of baseline biological information stored in the patient database 24;
initiating, by the emergency response system server 21, an emergency call to a nearby emergency dispatch center 26 to notify a live emergency responder 262 with the patient's GPS location information and patient information including contact information of patient's relatives and friends on record;
connecting, by the live emergency responder 262, to one or more patient's relatives and friends on record and a nearby medical facility to coordinate immediate medical assistance to the patient 30 based on the patient information received and retrieved from the patient database 24;
providing, by the live emergency responder 262 and the emergency response system server 21, patient specific medical assistance instructions for the patient 30 to follow including instructing the patient 30 to take one or more patient specific emergency medicines stored in an emergency medicine storage 108 of the comprehensive patient biological information collection device 10; and
communicating, by the patient 30 through a communication system 23 of the emergency response system 20, with the live emergency responder 262 and the one or more patient's relatives and friends on record until an ambulance from a nearby medical facility arrives.

FIG. 9 shows a flow chart of the method 900 of using the emergency response system 20 having a set of comprehensive patient biological information collection devices 10 according to certain embodiments of the present disclosure.

At block 902, each of a group of patients 30 registers the patient himself/herself, and one corresponding comprehensive patient biological information collection device at an emergency response system server 21 of the emergency response system 20, collecting, a set of baseline biological information for each patient 30, and storing the baseline biological information collected in the patient database 24, patient information of each of the patients 30 is stored in the patient database 24 of the emergency response system 20, and the of the emergency response system 20 provide immediate emergency assistance to each of the patients 30 when a comprehensive patient biological information collection device 10 detects one or more emergency signs from a corresponding patient 30. The emergency response system 20 includes a network of emergency dispatch centers 26 to provide immediate emergency assistance to the patients 30 when emergencies occur to the registered patients 30.

At block 904, each of the comprehensive patient biological information collection device 10 is activated at the emergency response system server 21 of the emergency response system 20, and each of the comprehensive patient biological information collection devices 10 monitors biological information of each patient 30 through a set of patient biological information collection sensors 400 of the comprehensive patient biological information collection device 10. In certain embodiments, the patient biological information includes: speech samples, facial images and video, electroencephalography (EEG) data, electrocardiography (ECG/EKG) data, breath samples, blood glucose content, blood oxygen content, pulse rate, blood pressure, sound samples such as breathing, heart, thoracic, arterial, intravenous, uterine, fetal, intestinal and other sounds, and body temperatures at various time.

At block 906, the biological information of each patient 30 collected by the set of patient biological information collection sensors 400 is transmitted to the emergency response system server 21 of the emergency response system 20 through a communication network 22.

At block 908, the emergency response system server 21 processes and analyzes the live patient biological information received from each patient 30 using artificial intelligence algorithms and comparing the live patient biological information received from each patient 30 with corresponding patient baseline biological information stored in the patient database 24, and determines whether an emergency is about to happen to a patient 30.

When at least one of the patient biological information collection sensors 400 detects certain type of biological information that exceeds a normal range of the set of baseline biological information of the patient 30 stored in the patient database 24, the emergency response system server 21 initiates at least voice communication between the emergency response system server 21 and the comprehensive patient biological information collection device 10 of the patient 30 through the communication system 23 directly to notify the patient 30 that there is high possibility that an medical emergency is about to happen to the patient 30.

The emergency response system server 21 initiates an emergency call to a nearby emergency dispatch center 26 to notify a live emergency responder 262 with the patient's GPS location information and patient information including contact information of patient's relatives, friends and other responders on record. The live emergency responder 262 connects to one or more patient's relatives, friends and other responders on record and a nearby medical facility to coordinate immediate medical assistance to the patient 30 based on the patient information received and retrieved from the patient database 24.

At query block 910, based on the severity of the emergency, patient medical history, and other patient information, the artificial intelligence algorithms of the emergency response system server 21, the live emergency responder 262, the patient 30, as well as one or more patient's relatives, friends and other responders on record determine whether an ambulance is needed for this emergency. When the answer is yes, the method 900 proceeds to block 912 to coordinate the arrangement of the ambulance from nearby medical emergency facilities. Otherwise, the method 900 proceeds to block 914 to coordinate the patient 30 to help himself/herself without the ambulance.

At block 912, when the ambulance is needed, the live emergency responder 262 notifies the nearby emergency medical facility to send the ambulance and patient specific professional staffs to assist the patient 30. In the meantime, patient specific medical assistance instructions for the patient 30 to follow are sent to the patient 30. These patient specific medical assistance instructions include necessary steps the patient 30 can perform and instructions to take one or more patient specific emergency medicines stored in the emergency medicine storage 108 of the comprehensive patient biological information collection device 10 of the patient 30, until the ambulance and the patient specific professional staffs arrive.

At block 914, when the ambulance is not needed, patient specific medical assistance instructions for the patient 30 to follow are sent to the patient 30. These patient specific medical assistance instructions include necessary steps the patient 30 can perform and instructions to take one or more patient specific emergency medicines stored in the emergency medicine storage 108 of the comprehensive patient biological information collection device 10. The patient 30 stays online with the live emergency responder 262, the patient's relatives, friends and other responders on record, and emergency medical staff until the emergency is over.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain principles of this invention and their practical application to enable others skilled in the art to utilize this invention and various embodiments and with various modifications as are suited to the particular use contemplated. Alternative embodiments will become apparent to those skilled in the art to which present disclosure pertains without departing from its spirit and scope. Accordingly, the scope of the present disclosure is defined by appended claims rather than the foregoing description and exemplary embodiments described therein.

What is claimed is:

1. A comprehensive patient biological information collection device comprises:
a processor, wherein the processor controls operations of the comprehensive patient biological information collection device;
a network interface controller, wherein the network interface controller facilitates the communication among the comprehensive patient biological information collection device, and a plurality of patient biological information collection sensors over a communication network; and
a non-volatile memory, wherein the non-volatile memory stores an operating system, a GPS module for detecting the GPS location of the patient carrying the comprehensive patient biological information collection device, and a comprehensive patient biological information collection device controller having a patient communication module, a comprehensive patient biological information collection module, a comprehensive patient biological information processing module, a comprehensive patient biological information storage module; and computer executable instructions, when executed by the processor, the computer executable instructions perform one or more of following operations:
receiving, through the comprehensive patient biological information collection module over the patient communication module, patient biological information constantly monitored and collected by a plurality of patient biological information collection sensors of the comprehensive patient biological information collection device;
processing, by the comprehensive patient biological information processing module, the patient biological information received from the plurality of patient biological information collection sensors;
storing, by the comprehensive patient biological information storage module, the processed patient biological information; and
updating, by the patient communication module, a patient database.

2. The comprehensive patient biological information collection device according to claim 1, wherein
the patient communication module facilitates communication among the patient database, the comprehensive patient biological information collection device controller, and the plurality of patient biological information collection sensors through the network interface controller over the communication network;
the comprehensive patient biological information collection module collects patient biological information through a wireless data receiver over the communication network from the plurality of patient biological information collection sensors from the patient;
the comprehensive patient biological information processing module processes the patient biological information collected through the comprehensive patient biological information collection module; and
the comprehensive patient biological information storage module stores the patient biological information processed through the comprehensive patient biological information processing module and synchronizes the patient biological information stored with the patient database.

3. The comprehensive patient biological information collection device according to claim 1, wherein the communication network comprises a wireless personal area network (WPAN) having a Wi-Fi network, a Bluetooth network, an infrared network, and a Zigbee network, a wireless local area network (WLAN), a wireless metropolitan area network (WMAN), a wireless wide area network (WWAN), a cellular network, and a mobile communication network.

4. The comprehensive patient biological information collection device according to claim 3, wherein the plurality of patient biological information collection sensors comprises one or more of:
a chip reader, wherein the chip reader reads a patient information card where patient information is stored;
a geolocation sensor, wherein the geolocation sensor monitors the geolocation of the patient;
a speaker, wherein the speaker is used for the patient to make and receive voice calls, and to provide voice instructions to the patient;
a stethoscope sensor, wherein the stethoscope sensor monitors patient's heart, lung and breathing conditions;
a blood oxygen sensor, wherein the blood oxygen sensor monitors patient's blood oxygen level;
a motion sensor, wherein the motion sensor monitors patient's movement;
an acoustic sensor, wherein the acoustic sensor is used by the patient to make and receive voice calls, to generate voice samples of the patient, and to detect background sounds surrounding the patient;
a temperature sensor, wherein the temperature sensor monitors patient's body temperature;
a blood pressure sensor, wherein the blood pressure sensor monitors patient's blood pressure;
a blood sugar sensor, wherein the blood sugar sensor monitors patient's blood sugar level;
an EKG/ECG sensor, wherein the EKG/ECG sensor monitors patient's electrocardiograms;
an EEG sensor, wherein the EEG sensor monitors patient's electroencephalograms;
a video camera, wherein the EEG sensor monitors patient's posture, movement, facial and emotional expressions;
an accelerometer, wherein the accelerometer detects impact to the patient and sudden fall of the patient;
an abnormal gas sensor(s), wherein the abnormal gas sensor detects harmful gas, around the patient;
a breathalyzer sensor, wherein the breathalyzer sensor detects blood alcohol level of the patient; and
any combinations thereof.

5. An emergency response system, comprising:
an emergency response system server, wherein each of a plurality of patients and a corresponding comprehensive patient biological information collection device each patient carries are registered at the emergency response system server, and the emergency response system provides immediate emergency assistance to a patient when a comprehensive patient biological information collection device of the patient detects one or more emergency signs from the patient;
a patient database connected to and accessible by the emergency response system server, wherein the patient database stores patient information of the plurality of patients, wherein the patient information comprises personal information, medical history, a set of baseline biological information, patient contact information, and contact information of relatives and friends and local medical facilities to be notified;
a communication system connected to the emergency response system server, wherein the communication system provides voice, text, and video communication over a communication network among the patient, one or more live emergency responders from a nearby emergency dispatch center, one or more patient's relatives and friends on record, and one or more nearby medical facilities to provide immediate emergency assistance to the patient when emergency occurs; and
a plurality of comprehensive patient biological information collection devices, wherein each patient carries a corresponding comprehensive patient biological information collection device, and each comprehensive patient biological information collection device collects patients' biological information through a plurality of patient biological information collection sensors and the plurality of patient biological information collection sensors constantly monitors the biological information of the patient and transmits the patient biological information collected to the emergency response system server through the comprehensive patient biological information collection device, and when at least one of the patient biological information collection sensors detects certain type of biological information that exceeds a normal range of the set of baseline biological information stored in the patient database, the emergency response system server initiates at least voice communication between the emergency response system server and the comprehensive patient biological information collection device of the patient through the communication system directly; and
an emergency medicine storage for storing one or more patient specific emergency medicines to be used when emergency occurs,
wherein a patient emergency indicator of the comprehensive patient biological information collection device is lit in green light indicating the comprehensive patient biological information collection device is in normal operation state, and the patient is in normal condition, when an emergency sign is detected from the patient, the patient emergency indicator turns red indicating the patient is in an emergency, the comprehensive patient biological information collection device initiates an emergency call to the nearby emergency dispatch center to notify a live emergency responder with the patient's GPS location information and patient information including contact information of patient's relatives and friends on record, the live emergency responder connects to one or more patient's relatives and friends on record and a nearby medical facility to coordinate immediate medical assistance to the patient based on the patient information received and retrieved from the patient database, and the live emergency responder and the emergency response system server provide patient specific medical assistance instructions for the patient to follow including instructing the patient to take one or more patient specific emergency medicines stored in the emergency medicine storage of the comprehensive patient biological information collection device, and the patient continues to communicate with the live emergency responder and the one or more patient's relatives and friends on record until an ambulance from a nearby medical facility arrives.

6. The emergency response system according to claim 5, wherein the communication network comprises a wireless personal area network (WPAN) having a Wi-Fi network, a Bluetooth network, an infrared network, and a Zigbee network, a wireless local area network (WLAN), a wireless metropolitan area network (WMAN), a wireless wide area network (WWAN), a cellular network, and a mobile communication network.

7. The emergency response system according to claim 5, wherein the emergency response system server comprises:
a server processor for controlling operations of the emergency response system;
a network interface controller connected to the communication network through a firewall connector over a firewall; and
a non-volatile memory for storing an server operating system, a network communication module, and an emergency response controller having a patient information storage module for accessing the patient database through a database interface, a communication control module for facilitating communication to the communication system through a communication control interface, a patient information processing module, and computer executable instructions, when executed by the server processor, the computer executable instructions perform one or more of following operations:
receiving, through a patient communication module of the comprehensive patient biological information collection device of the patient, patient biological information constantly monitored and collected by the plurality of patient biological information collection sensors of the comprehensive patient biological information collection device;
processing, by the patient information processing module, the patient biological information received;
comparing, by the patient information processing module, the patient biological information processed against base line patient biological information from the patient database;
initiating, by the emergency response system server, an emergency voice call to the comprehensive patient biological information collection device of the patient through the communication system, when at least one of the patient biological information collection sensors detects certain type of biological information that exceeds a normal range of the set of baseline biological information stored in the patient database;
initiating, by the emergency response system server, an emergency call to the nearby emergency dispatch center to notify the live emergency responder with the patient's GPS location information and patient information including contact information of patient's relatives and friends on record;
connecting, through the communication control module, the comprehensive patient biological information collection device carried by the patient to the patient's relatives and friends on record and a nearby medical facility to coordinate immediate medical assistance to the patient; and
transmitting, through the communication control module, a set of patient specific medical assistance instructions through the communication control interface and the live emergency responder for the patient to follow including instructing the patient to take one or more patient specific emergency medicines stored in the emergency medicine storage of the comprehensive patient biological information collection device, and the patient continues to communicate with the live emergency responder and the one or more patient's relatives and friends on record until an ambulance from the nearby medical facility arrives.

8. The emergency response system according to claim 5, wherein each of the plurality of comprehensive patient biological information collection devices comprises:
a processor, wherein the processor controls operations of the comprehensive patient biological information collection device;
a network interface controller, wherein the network interface controller facilitates the communication among the comprehensive patient biological information collection device, the emergency response system server and the communication system over the communication network; and
a non-volatile memory, wherein the non-volatile memory stores an operating system, a GPS module for detecting the GPS location of the patient carrying the comprehensive patient biological information collection device, and a comprehensive patient biological information collection device controller having the patient communication module, a comprehensive patient biological information collection module, a comprehensive patient biological information processing module, a comprehensive patient biological information storage module; and computer executable instructions, when executed by the processor, the computer executable instructions perform one or more of following operations:
receiving, through the comprehensive patient biological information collection module over the patient communication module, patient biological information constantly monitored and collected by a plurality of patient biological information collection sensors;
processing, by the comprehensive patient biological information processing module, the patient biological information received from the plurality of patient biological information collection sensors;
storing, by the comprehensive patient biological information storage module, the processed patient biological information;
and updating, by the patient communication module, a patient database.

9. The emergency response system according to claim 8, wherein the plurality of patient biological information collection sensors comprises one or more of:
a chip reader, wherein the chip reader reads a patient information card where patient information is stored;
a geolocation sensor, wherein the geolocation sensor monitors the geolocation of the patient;
a speaker, wherein the speaker is used for the patient to make and receive voice calls, and to provide voice instructions to the patient;
a stethoscope sensor, wherein the stethoscope sensor monitors patient's heart, lung and breathing conditions;
a blood oxygen sensor, wherein the blood oxygen sensor monitors patient's blood oxygen level;
a motion sensor, wherein the motion sensor monitors patient's movement;
an acoustic sensor, wherein the acoustic sensor is used by the patient to make and receive voice calls, to generate voice samples of the patient, and to detect background sounds surrounding the patient;
a temperature sensor, wherein the temperature sensor monitors patient's body temperature;
a blood pressure sensor, wherein the blood pressure sensor monitors patient's blood pressure;
a blood sugar sensor, wherein the blood sugar sensor monitors patient's blood sugar level;
an EKG/ECG sensor, wherein the EKG/ECG sensor monitors patient's electrocardiograms;
an EEG sensor, wherein the EEG sensor monitors patient's electroencephalograms;
a video camera, wherein the EEG sensor monitors patient's posture, movement, facial and emotional expressions;
an accelerometer, wherein the accelerometer detects impact to the patient and sudden fall of the patient;
an abnormal gas sensor, wherein the abnormal gas sensor detects harmful gas around the patient;
a breathalyzer sensor, wherein the breathalyzer sensor detects blood alcohol level of the patient; and
any combinations thereof.

10. The emergency response system according to claim 5, wherein the comprehensive patient biological information collection device further comprises:
an emergency button, wherein the patient presses the emergency button to notify the emergency response system when an emergency occurs;
the patient emergency indicator; wherein the patient emergency indicator is lit in green indicating that the patient is in normal condition, and the patient emergency indicator turns in red indicating that the patient is in an emergency condition;
a display screen, wherein the display screen receives and displays text messages, emails, emergency assistance messages, and carries out video calls;
a power supply module, wherein the power supply module provides electrical power to the comprehensive patient biological information collection device;
the emergency medicine storage, wherein one or more patient specific emergency medicines for the patient are stored; and
a SIM card slot, wherein a SIM card for mobile communication is inserted in the SIM card slot to enable wireless communication.

11. The emergency response system according to claim 5, wherein the communication among the patient, the live emergency responder, the patient's relatives and friends on record and the nearby medical facility comprises:
mobile voice calls;
mobile videotelephony calls;
landline voice calls;
videotelephony calls over the Internet;
text messages over a mobile phone;
text messages over a plurality of social media platforms; and
videotelephony calls over the plurality of social media platforms.

12. The emergency response system according to claim 11, wherein the communication system comprises:
a voice input module having a mobile voice input interface to receive mobile voice calls, and a landline voice input interface to receive landline voice calls;
a text input module having a social media text input interface to receive text messages through the plurality of social media platforms, and a text message input interface to receive text messages through mobile phones;
a video input module having a mobile video input interface to receive video calls over the mobile phones, and a social media video input interface to receive video calls through the plurality of social media platforms;
a voice output module having a mobile voice output interface to make mobile voice calls, and a landline voice output interface to make landline voice calls;
a text output module having a social media text output interface to transmit text messages through the plurality of social media platforms, and a text message output interface to transmit text messages through the mobile phones;
a video output module having a mobile video output interface to make video calls over the mobile phones, and a social media video output interface to make video calls through the plurality of social media platforms;
a speech to text conversion module for converting voice input to text input;
a live emergency responder interface module for the live emergency responder to receive and make conference calls among the patient, the live emergency responder, the patient's relatives and friends on record and the nearby medical facility through voice calls, text messages, and video calls; and
a text to speech conversion module to make voice calls to the patient through the comprehensive patient biological information collection device.

13. The emergency response system according to claim 7, wherein the communication control interface comprises:
a voice input terminal connected to the voice input module;
a text input terminal connected to the text input module;
a video input terminal connected to the video input module;
a voice output terminal connected to the voice output module;
a text output terminal connected to the text output module; and
a video output terminal connected to the video output module.

14. The emergency response system according to claim 5, wherein the comprehensive patient biological information collection device comprises:
a portable patient alert device to be carried with the patient wherein the portable patient alert device stores one or more patient specific emergency medicines;
a stationary patient alert device to be placed at home or work place of the patient wherein the stationary patient alert device stores one or more patient specific emergency medicines; and
a plurality of public stationary patient alert devices to be placed in public places wherein each of the plurality of public stationary patient alert devices stores at least one of a plurality of common emergency medicines.

15. A method of using an emergency response system, comprising:
registering, by a plurality of patients, each of the plurality of patients, and a plurality of comprehensive patient biological information collection devices, one corresponding comprehensive patient biological information collection device for each patient, at an emergency response system server of the emergency response system, wherein patient information of each of the plurality of patients is stored in a patient database of the emergency response system, and the emergency response system provide immediate emergency assistance to a patient when a comprehensive patient biological information collection device of the patient detects one or more emergency signs from the patient;
constantly monitoring and collecting, by a plurality of patient biological information collection sensors of the plurality of the comprehensive patient biological information collection devices, patient biological information of each of the plurality of patients;
receiving, through a patient communication control module 10664 from the comprehensive patient biological information collection device, the patient biological information constantly monitored and collected by the plurality of patient biological information collection sensors of the comprehensive patient biological information collection device;
processing, through a patient information processing module of the emergency response system server of the emergency response system, the patient biological information received, and comparing the patient biological information received with a set of baseline biological information stored in the patient database;
initiating, by the emergency response system server, at least voice communication between the emergency response system server and the comprehensive patient biological information collection device of the patient through the communication system directly, when at least one of the patient biological information collection sensors detects certain type of biological information that exceeds a normal range of the set of baseline biological information stored in the patient database;
initiating, by the emergency response system server, an emergency call to a nearby emergency dispatch center to notify a live emergency responder with the patient's GPS location information and patient information including contact information of patient's relatives and friends on record;
connecting, by the live emergency responder, to one or more patient's relatives and friends on record and a nearby medical facility to coordinate immediate medical assistance to the patient based on the patient information received and retrieved from the patient database;
providing, by the live emergency responder and the emergency response system server, patient specific medical assistance instructions for the patient to follow including instructing the patient to take one or more patient specific emergency medicines stored in an emergency medicine storage of the comprehensive patient biological information collection device; and
communicating, by the patient through a communication system of the emergency response system, with the live emergency responder and the one or more patient's relatives and friends on record until an ambulance from a nearby medical facility arrives.

16. The method of claim 15, wherein the emergency response system comprises:
the emergency response system server, wherein each of the plurality of patients and a corresponding comprehensive patient biological information collection device each patient carries are registered at the emergency response system server, and the emergency response system server provides immediate emergency assistance to each of the plurality of patients when emergencies occur;
the patient database connected to and accessible by the emergency response system server, wherein the patient database stores patient information of the plurality of patients, wherein the patient information comprises personal information, medical history, patient contact information, and contact information of relatives and friends on record and local medical facilities to be notified of each of the plurality of the patients;
the communication system connected to the emergency response system server, wherein the communication system provides voice, text, and video communication over a communication network among the patient, one or more live emergency responders from the nearby emergency dispatch center, one or more patient's relatives and friends on record, and one or more nearby medical facilities to provide immediate emergency assistance to the patient when emergency occurs; and
the plurality of comprehensive patient biological information collection devices, wherein each of the plurality of patients carries one corresponding comprehensive patient biological information collection device, and each comprehensive patient biological information collection device collects patients' biological information through the set of patient biological information collection sensors and the set of patient biological information collection sensors constantly monitors the biological information of the patient and transmits the patient biological information collected to the emergency response system server through the comprehensive patient biological information collection device, when at least one of the patient biological information collection sensors detects certain type of biological information that exceeds a normal range of the set of baseline biological information stored in the patient database, the emergency response system server initiates at least voice communication between the emergency response system server and the comprehensive patient biological information collection device of the patient through the communication system directly, and the plurality comprehensive patient biological information collection device also comprises an emergency medicine storage for storing one or more patient specific emergency medicines to be used when emergency occurs.

17. The method of claim 16, wherein the communication network comprises a wireless personal area network (WPAN) having a Wi-Fi network, a Bluetooth network, an infrared network, and a Zigbee network, a wireless local area network (WLAN), a wireless metropolitan area network (WMAN), a wireless wide area network (WWAN), a cellular network, and a mobile communication network.

18. The method of claim 17, wherein the comprehensive patient biological information collection device comprises:
a processor, wherein the processor controls operations of the comprehensive patient biological information collection device;
a network interface controller, wherein the network interface controller facilitates the communication among the comprehensive patient biological information collection device, the emergency response system server and the communication system over the communication network; and
a non-volatile memory, wherein the non-volatile memory stores an operating system, a GPS module for detecting the GPS location of the patient carrying the comprehensive patient biological information collection device, and a comprehensive patient biological information collection device controller having a patient communication module, a comprehensive patient biological information collection module, a comprehensive patient biological information processing module, a comprehensive patient biological information storage module; and computer executable instructions, when executed by the processor, the computer executable instructions perform one or more of following operations:
receiving, through the comprehensive patient biological information collection module over the patient communication module, patient biological information constantly monitored and collected by a plurality of patient biological information collection sensors of the comprehensive patient biological information collection device;
processing, by the comprehensive patient biological information processing module, the patient biological information received from the plurality of patient biological information collection sensors;
storing, by the comprehensive patient biological information storage module, the processed patient biological information;
and updating, by the patient communication module, the patient database of the emergency response system.

19. The method of claim 18, wherein the plurality of patient biological information collection sensors comprises one or more of:
a chip reader, wherein the chip reader reads a patient information card where patient information is stored;
a geolocation sensor, wherein the geolocation sensor monitors the geolocation of the patient;
a speaker, wherein the speaker is used for the patient to make and receive voice calls, and to provide voice instructions to the patient;
a stethoscope sensor, wherein the stethoscope sensor monitors patient's heart, lung and breathing conditions;
a blood oxygen sensor, wherein the blood oxygen sensor monitors patient's blood oxygen level;
a motion sensor, wherein the motion sensor monitors patient's movement;
an acoustic sensor, wherein the acoustic sensor is used by the patient to make and receive voice calls, to generate voice samples of the patient, and to detect background sounds surrounding the patient;
a temperature sensor, wherein the temperature sensor monitors patient's body temperature;
a blood pressure sensor, wherein the blood pressure sensor monitors patient's blood pressure;
a blood sugar sensor, wherein the blood sugar sensor monitors patient's blood sugar level;
an EKG/ECG sensor, wherein the EKG/ECG sensor monitors patient's electrocardiograms;
an EEG sensor, wherein the EEG sensor monitors patient's electroencephalograms;
a video camera, wherein the EEG sensor monitors patient's posture, movement, facial and emotional expressions;
an accelerometer, wherein the accelerometer detects impact to the patient and sudden fall of the patient;
an abnormal gas sensor, wherein the abnormal gas sensor detects harmful gas around the patient;
a breathalyzer sensor, wherein the breathalyzer sensor detects blood alcohol level of the patient; and
any combinations thereof.

20. The method of claim 19, wherein the comprehensive patient biological information collection device comprises:
a portable patient alert device to be carried with the patient wherein the portable patient alert device stores one or more patient specific emergency medicines;
a stationary patient alert device to be placed at home or work place of the patient wherein the stationary patient alert device stores one or more patient specific emergency medicines; and
a plurality of public stationary patient alert devices to be placed in public places wherein each of the plurality of public stationary patient alert devices stores at least one of a plurality of common emergency medicines.
